SEQUENCE LISTING (114 sequences)

SEQ ID NO: 1 - DNA, 50 bp, Unknown
  source: /note=“Description of Unknown Wild type Mu transposon end sequence, transferred strand”
gttttcgcatttatcgtgaaacgctttcgcgtttttcgtgcgccgcttca

SEQ ID NO: 2 - DNA, 54 bp, Unknown
  source: /note=“Description of Unknown Wild type Mu transposon end sequence, non-transferred strand”
ctagtgaagcggcgcacgaaaaacgcgaaagcgtttcacgataaatgcgaaaac

SEQ ID NO: 3 - DNA, 70 bp, Artificial Sequence
  source: /note=“Description of Artificial Sequence Synthetic oligonucleotide”
gttttcgcatttatcgtgaaacgctttcgcgtttttcgtgcgccgcttcactagtttggcgtaatcgccg

SEQ ID NO: 4 - DNA, 70 bp, Artificial Sequence
  source: /note=“Description of Artificial Sequence Synthetic oligonucleotide”
  modified_base (45)..(49): a, c, g, or t
gttttcgcatttatcgtgaaacgctttcgcgtttttcgtgcgccnnnnnactagtttggcgtaatcgccg

SEQ ID NO: 5 - DNA, 70 bp, Artificial Sequence
  source: /note=“Description of Artificial Sequence Synthetic oligonucleotide”
  modified_base (35)..(38): a, c, g, or t
  modified_base (41)..(43): a, c, g, or t
  modified_base (45)..(49): a, c, g, or t
gttttcgcatttatcgtgaaacgctttcgcgtttnnnntgnnncnnnnnactagtttggcgtaatcgccg

SEQ ID NO: 6 - DNA, 70 bp, Artificial Sequence
  source: /note=“Description of Artificial Sequence Synthetic oligonucleotide”
  modified_base (1)..(2): a, c, g, or t
  modified_base (8)..(10): a, c, g, or t
  modified_base (13)..(16): a, c, g, or t
  modified_base (19)..(21): a, c, g, or t
  modified_base (23)..(24): a, c, g, or t
  modified_base (30)..(32): a, c, g, or t
  modified_base (35)..(38): a, c, g, or t
  modified_base (41)..(43): a, c, g, or t
  modified_base (45)..(49): a, c, g, or t
nntttcgnnnttnnnntgnnncnntttcgnnnttnnnntgnnncnnnnnactagtttggcgtaatcgccg

SEQ ID NO: 7 - DNA, 22 bp, Artificial Sequence
  source: /note=“Description of Artificial Sequence Synthetic oligonucleotide”
cggcgattacgccaaactagtg

SEQ ID NO: 8 - DNA, 70 bp, Artificial Sequence; identical to SEQ ID NO: 3
  source: /note=“Description of Artificial Sequence Synthetic oligonucleotide”

SEQ ID NO: 9 - DNA, 70 bp, Artificial Sequence
  source: /note=“Description of Artificial Sequence Synthetic oligonucleotide”
cggcgattacgccaaactagtgaagcggcgcacgaaaaacgcgaaagcgtttcacgataaatgcgaaaac

SEQ ID NO: 10 - DNA, 70 bp, Artificial Sequence; identical to SEQ ID NO: 4
  source: /note=“Description of Artificial Sequence Synthetic oligonucleotide”
  modified_base (45)..(49): a, c, g, or t

SEQ ID NO: 11 - DNA, 70 bp, Artificial Sequence
  source: /note=“Description of Artificial Sequence Synthetic oligonucleotide”
  modified_base (22)..(26): a, c, g, or t
cggcgattacgccaaactagtnnnnnggcgcacgaaaaacgcgaaagcgtttcacgataaatgcgaaaac

SEQ ID NO: 12 - DNA, 50 bp, Artificial Sequence
  source: /note=“Description of Artificial Sequence Synthetic oligonucleotide”
  modified_base (45)..(49): a, c, g, or t
gttttcgcatttatcgtgaaacgctttcgcgtttttcgtgcgccnnnnna

SEQ ID NO: 13 - DNA, 54 bp, Artificial Sequence
  source: /note=“Description of Artificial Sequence Synthetic oligonucleotide”
  modified_base (6)..(10): a, c, g, or t
ctagtnnnnnggcgcacgaaaaacgcgaaagcgtttcacgataaatgcgaaaac

SEQ ID NO: 14 - DNA, 70 bp, Artificial Sequence; identical to SEQ ID NO: 5
  source: /note=“Description of Artificial Sequence Synthetic oligonucleotide”
  modified_base (35)..(38): a, c, g, or t
  modified_base (41)..(43): a, c, g, or t
  modified_base (45)..(49): a, c, g, or t

SEQ ID NO: 15 - DNA, 70 bp, Artificial Sequence
  source: /note=“Description of Artificial Sequence Synthetic oligonucleotide”
  modified_base (22)..(26): a, c, g, or t
  modified_base (28)..(30): a, c, g, or t
  modified_base (33)..(36): a, c, g, or t
cggcgattacgccaaactagtnnnnngnnncannnnaaacgcgaaagcgtttcacgataaatgcgaaaac

SEQ ID NO: 16 - DNA, 50 bp, Artificial Sequence
  source: /note=“Description of Artificial Sequence Synthetic oligonucleotide”
  modified_base (35)..(38): a, c, g, or t
  modified_base (41)..(43): a, c, g, or t
  modified_base (45)..(49): a, c, g, or t
gttttcgcatttatcgtgaaacgctttcgcgtttnnnntgnnncnnnnna

SEQ ID NO: 17 - DNA, 54 bp, Artificial Sequence
  source: /note=“Description of Artificial Sequence Synthetic oligonucleotide”
  modified_base (6)..(10): a, c, g, or t
  modified_base (12)..(14): a, c, g, or t
  modified_base (17)..(20): a, c, g, or t
ctagtnnnnngnnncannnnaaacgcgaaagcgtttcacgataaatgcgaaaac

SEQ ID NO: 18 - DNA, 70 bp, Artificial Sequence; identical to SEQ ID NO: 6
  source: /note=“Description of Artificial Sequence Synthetic oligonucleotide”
  modified_base (1)..(2): a, c, g, or t
  modified_base (8)..(10): a, c, g, or t
  modified_base (13)..(16): a, c, g, or t
  modified_base (19)..(21): a, c, g, or t
  modified_base (23)..(24): a, c, g, or t
  modified_base (30)..(32): a, c, g, or t
  modified_base (35)..(38): a, c, g, or t
  modified_base (41)..(43): a, c, g, or t
  modified_base (45)..(49): a, c, g, or t

SEQ ID NO: 19 - DNA, 70 bp, Artificial Sequence
  source: /note=“Description of Artificial Sequence Synthetic oligonucleotide”
  modified_base (22)..(26): a, c, g, or t
  modified_base (28)..(30): a, c, g, or t
  modified_base (33)..(36): a, c, g, or t
  modified_base (39)..(41): a, c, g, or t
  modified_base (47)..(48): a, c, g, or t
  modified_base (50)..(52): a, c, g, or t
  modified_base (55)..(58): a, c, g, or t
  modified_base (61)..(63): a, c, g, or t
  modified_base (69)..(70): a, c, g, or t
cggcgattacgccaaactagtnnnnngnnncannnnaannncgaaanngnnncannnnaannncgaaann

SEQ ID NO: 20 - DNA, 50 bp, Artificial Sequence
  source: /note=“Description of Artificial Sequence Synthetic oligonucleotide”
  modified_base (1)..(2): a, c, g, or t
  modified_base (8)..(10): a, c, g, or t
  modified_base (13)..(16): a, c, g, or t
  modified_base (19)..(21): a, c, g, or t
  modified_base (23)..(24): a, c, g, or t
  modified_base (30)..(32): a, c, g, or t
  modified_base (35)..(38): a, c, g, or t
  modified_base (41)..(43): a, c, g, or t
  modified_base (45)..(49): a, c, g, or t
nntttcgnnnttnnnntgnnncnntttcgnnnttnnnntgnnncnnnnna

SEQ ID NO: 21 - DNA, 54 bp, Artificial Sequence
  source: /note=“Description of Artificial Sequence Synthetic oligonucleotide”
  modified_base (6)..(10): a, c, g, or t
  modified_base (12)..(14): a, c, g, or t
  modified_base (17)..(20): a, c, g, or t
  modified_base (23)..(25): a, c, g, or t
  modified_base (31)..(32): a, c, g, or t
  modified_base (34)..(36): a, c, g, or t
  modified_base (39)..(42): a, c, g, or t
  modified_base (45)..(47): a, c, g, or t
  modified_base (53)..(54): a, c, g, or t
ctagtnnnnngnnncannnnaannncgaaanngnnncannnnaannncgaaann

SEQ ID NO: 22 - DNA, 50 bp, Artificial Sequence
  source: /note=“Description of Artificial Sequence Synthetic oligonucleotide”
gttttcgcatttatcgtgaaacgctttcgcgtttttcgtgcgcctacgca

SEQ ID NO: 23 - DNA, 54 bp, Artificial Sequence
  source: /note=“Description of Artificial Sequence Synthetic oligonucleotide”
ctagtgcgtaggcgcacgaaaaacgcgaaagcgtttcacgataaatgcgaaaac

SEQ ID NO: 24 - DNA, 50 bp, Artificial Sequence
  source: /note=“Description of Artificial Sequence Synthetic oligonucleotide”
gttttcgcatttatcgtgaaacgctttcgcgtttttcgtgcgccgtacca

SEQ ID NO: 25 - DNA, 54 bp, Artificial Sequence
  source: /note=“Description of Artificial Sequence Synthetic oligonucleotide”
ctagtggtacggcgcacgaaaaacgcgaaagcgtttcacgataaatgcgaaaac

SEQ ID NO: 26 - DNA, 50 bp, Artificial Sequence
  source: /note=“Description of Artificial Sequence Synthetic oligonucleotide”
gttttcgcatttatcgtgaaacgctttcgcgtttttcgtgcgcccgcaca

SEQ ID NO: 27 - DNA, 54 bp, Artificial Sequence
  source: /note=“Description of Artificial Sequence Synthetic oligonucleotide”
ctagtgtgcgggcgcacgaaaaacgcgaaagcgtttcacgataaatgcgaaaac

SEQ ID NO: 28 - DNA, 50 bp, Artificial Sequence
  source: /note=“Description of Artificial Sequence Synthetic oligonucleotide”
gttttcgcatttatcgtgaaacgctttcgcgtttttcgtgcgccacatca

SEQ ID NO: 29 - DNA, 54 bp, Artificial Sequence
  source: /note=“Description of Artificial Sequence Synthetic oligonucleotide”
ctagtgatgtggcgcacgaaaaacgcgaaagcgtttcacgataaatgcgaaaac

SEQ ID NO: 30 - DNA, 50 bp, Artificial Sequence
  source: /note=“Description of Artificial Sequence Synthetic oligonucleotide”
gttttcgcatttatcgtgaaacgctttcgcgtttttcgtgcgcccatgca

SEQ ID NO: 31 - DNA, 54 bp, Artificial Sequence
  source: /note=“Description of Artificial Sequence Synthetic oligonucleotide”
ctagtgcatgggcgcacgaaaaacgcgaaagcgtttcacgataaatgcgaaaac

SEQ ID NO: 32 - DNA, 50 bp, Artificial Sequence
  source: /note=“Description of Artificial Sequence Synthetic oligonucleotide”
gttttcgcatttatcgtgaaacgctttcgcgtttttcgtgcgcctttcca

SEQ ID NO: 33 - DNA, 54 bp, Artificial Sequence
  source: /note=“Description of Artificial Sequence Synthetic oligonucleotide”
ctagtggaaaggcgcacgaaaaacgcgaaagcgtttcacgataaatgcgaaaac

SEQ ID NO: 34 - DNA, 50 bp, Artificial Sequence
  source: /note=“Description of Artificial Sequence Synthetic oligonucleotide”
gttttcgcatttatcgtgaaacgctttcgcgtttttcgtgcgccgggaca

SEQ ID NO: 35 - DNA, 54 bp, Artificial Sequence
  source: /note=“Description of Artificial Sequence Synthetic oligonucleotide”
ctagtgtcccggcgcacgaaaaacgcgaaagcgtttcacgataaatgcgaaaac

SEQ ID NO: 36 - DNA, 50 bp, Artificial Sequence
  source: /note=“Description of Artificial Sequence Synthetic oligonucleotide”
gttttcgcatttatcgtgaaacgctttcgcgtttttcgtgcgccctctca

SEQ ID NO: 37 - DNA, 54 bp, Artificial Sequence
  source: /note=“Description of Artificial Sequence Synthetic oligonucleotide”
ctagtgagagggcgcacgaaaaacgcgaaagcgtttcacgataaatgcgaaaac

SEQ ID NO: 38 - DNA, 50 bp, Artificial Sequence
  source: /note=“Description of Artificial Sequence Synthetic oligonucleotide”
gttttcgcatttatcgtgaaacgctttcgcgtttttcgtgcgccagagca

SEQ ID NO: 39 - DNA, 54 bp, Artificial Sequence
  source: /note=“Description of Artificial Sequence Synthetic oligonucleotide”
ctagtgctctggcgcacgaaaaacgcgaaagcgtttcacgataaatgcgaaaac

SEQ ID NO: 40 - DNA, 50 bp, Artificial Sequence
  source: /note=“Description of Artificial Sequence Synthetic oligonucleotide”
gttttcgcatttatcgtgaaacgctttcgcgtttttcgtgcgccacgcca

SEQ ID NO: 41 - DNA, 54 bp, Artificial Sequence
  source: /note=“Description of Artificial Sequence Synthetic oligonucleotide”
ctagtggcgtggcgcacgaaaaacgcgaaagcgtttcacgataaatgcgaaaac

SEQ ID NO: 42 - DNA, 50 bp, Artificial Sequence
  source: /note=“Description of Artificial Sequence Synthetic oligonucleotide”
gttttcgcatttatcgtgaaacgctttcgcgtttttcgtgcgcctataca

SEQ ID NO: 43 - DNA, 54 bp, Artificial Sequence
  source: /note=“Description of Artificial Sequence Synthetic oligonucleotide”
ctagtgtataggcgcacgaaaaacgcgaaagcgtttcacgataaatgcgaaaac

SEQ ID NO: 44 - DNA, 50 bp, Artificial Sequence
  source: /note=“Description of Artificial Sequence Synthetic oligonucleotide”
gttttcgcatttatcgtgaaacgctttcgcgtttttcgtgcgccgcgtca

SEQ ID NO: 45 - DNA, 54 bp, Artificial Sequence
  source: /note=“Description of Artificial Sequence Synthetic oligonucleotide”
ctagtgacgcggcgcacgaaaaacgcgaaagcgtttcacgataaatgcgaaaac

SEQ ID NO: 46 - DNA, 19 bp, Artificial Sequence
  source: /note=“Description of Artificial Sequence Synthetic oligonucleotide”
agatgtgtataagagacag

SEQ ID NO: 47 - DNA, 13 bp, Artificial Sequence
  source: /note=“Description of Artificial Sequence Synthetic oligonucleotide”
gctcttccgatct

SEQ ID NO: 48 - DNA, 50 bp, Artificial Sequence
  source: /note=“Description of Artificial Sequence Synthetic oligonucleotide”
gttttagatgtgtataagagacagtttcgcgtttttcgtgcgccgcttca

SEQ ID NO: 49 - DNA, 54 bp, Artificial Sequence
  source: /note=“Description of Artificial Sequence Synthetic oligonucleotide”
ctagtgaagcggcgcacgaaaaacgcgaaactgtctcttatacacatctaaaac

SEQ ID NO: 50 - DNA, 50 bp, Artificial Sequence
  source: /note=“Description of Artificial Sequence Synthetic oligonucleotide”
gttttcgctcttccgatctaacgctttcgcgtttttcgtgcgccgcttca

SEQ ID NO: 51 - DNA, 54 bp, Artificial Sequence
  source: /note=“Description of Artificial Sequence Synthetic oligonucleotide”
ctagtgaagcggcgcacgaaaaacgcgaaagcgttagatcggaagagcgaaaac

SEQ ID NO: 52 - DNA, 50 bp, Artificial Sequence
  source: /note=“Description of Artificial Sequence Synthetic oligonucleotide”
gttttcgcatttatcgtgaaacgctttcgcgtttttgctcttccgatcta

SEQ ID NO: 53 - DNA, 54 bp, Artificial Sequence
  source: /note=“Description of Artificial Sequence Synthetic oligonucleotide”
ctagtagatcggaagagcaaaaacgcgaaagcgtttcacgataaatgcgaaaac

SEQ ID NO: 54 - DNA, 18 bp, Artificial Sequence
  source: /note=“Description of Artificial Sequence Synthetic oligonucleotide”
aattaaccctcactaaag

SEQ ID NO: 55 - DNA, 18 bp, Artificial Sequence
  source: /note=“Description of Artificial Sequence Synthetic oligonucleotide”
taatacgactcactatag

SEQ ID NO: 56 - DNA, 50 bp, Artificial Sequence
  source: /note=“Description of Artificial Sequence Synthetic oligonucleotide”
gttttcgaattaaccctcactaaagttcgcgtttttcgtgcgccgcttca

SEQ ID NO: 57 - DNA, 54 bp, Artificial Sequence
  source: /note=“Description of Artificial Sequence Synthetic oligonucleotide”
ctagtgaagcggcgcacgaaaaacgcgaactttagtgagggttaattcgaaaac

SEQ ID NO: 58 - DNA, 50 bp, Artificial Sequence
  source: /note=“Description of Artificial Sequence Synthetic oligonucleotide”
gttttcgcatttatcgtgaaacgctttcgcaattaaccctcactaaagca

SEQ ID NO: 59 - DNA, 54 bp, Artificial Sequence
  source: /note=“Description of Artificial Sequence Synthetic oligonucleotide”
ctagtgctttagtgagggttaattgcgaaagcgtttcacgataaatgcgaaaac

SEQ ID NO: 60 - DNA, 50 bp, Artificial Sequence
  source: /note=“Description of Artificial Sequence Synthetic oligonucleotide”
gttttcgcatttatcgtgaaacgctttcgcgaattaaccctcactaaaga

SEQ ID NO: 61 - DNA, 54 bp, Artificial Sequence
  source: /note=“Description of Artificial Sequence Synthetic oligonucleotide”
ctagtctttagtgagggttaattcgcgaaagcgtttcacgataaatgcgaaaac

SEQ ID NO: 62 - DNA, 50 bp, Artificial Sequence
  source: /note=“Description of Artificial Sequence Synthetic oligonucleotide”
gttttcgcatttaatacgactcactatagcgtttttcgtgcgccgcttca

SEQ ID NO: 63 - DNA, 54 bp, Artificial Sequence
  source: /note=“Description of Artificial Sequence Synthetic oligonucleotide”
ctagtgaagcggcgcacgaaaaacgctatagtgagtcgtattaaatgcgaaaac

SEQ ID NO: 64 - DNA, 50 bp, Artificial Sequence
  source: /note=“Description of Artificial Sequence Synthetic oligonucleotide”
gttttcgcatttatcgtgaaacgctttcgcgtaatacgactcactataga

SEQ ID NO: 65 - DNA, 54 bp, Artificial Sequence
  source: /note=“Description of Artificial Sequence Synthetic oligonucleotide”
ctagtctatagtgagtcgtattacgcgaaagcgtttcacgataaatgcgaaaac

SEQ ID NO: 66 - DNA, 50 bp, Artificial Sequence
  source: /note=“Description of Artificial Sequence Synthetic oligonucleotide”
  modified_base (1)..(2): a, c, g, or t
  modified_base (8)..(10): a, c, g, or t
  modified_base (13)..(16): a, c, g, or t
  modified_base (19)..(21): a, c, g, or t
  modified_base (23)..(24): a, c, g, or t
  modified_base (35)..(38): a, c, g, or t
  modified_base (41)..(43): a, c, g, or t
  modified_base (45)..(49): a, c, g, or t
nntttcgnnnttnnnntgnnncnntttcgcgtttnnnntgnnncnnnnna

SEQ ID NO: 67 - DNA, 50 bp, Artificial Sequence
  source: /note=“Description of Artificial Sequence Synthetic oligonucleotide”
  modified_base (1)..(2): a, c, g, or t
  modified_base (8)..(10): a, c, g, or t
  modified_base (13)..(16): a, c, g, or t
  modified_base (19)..(21): a, c, g, or t
  modified_base (23)..(24): a, c, g, or t
  modified_base (41)..(43): a, c, g, or t
  modified_base (45)..(49): a, c, g, or t
nntttcgnnnttnnnntgnnncnntttcgcgtttttcgtgnnncnnnnna

SEQ ID NO: 68 - DNA, 50 bp, Artificial Sequence
  source: /note=“Description of Artificial Sequence Synthetic oligonucleotide”
  modified_base (1)..(2): a, c, g, or t
  modified_base (8)..(10): a, c, g, or t
  modified_base (13)..(16): a, c, g, or t
  modified_base (19)..(21): a, c, g, or t
  modified_base (23)..(24): a, c, g, or t
  modified_base (45)..(49): a, c, g, or t
nntttcgnnnttnnnntgnnncnntttcgcgtttttcgtgcgccnnnnna

SEQ ID NO: 69 - DNA, 50 bp, Artificial Sequence
  source: /note=“Description of Artificial Sequence Synthetic oligonucleotide”
  modified_base (1)..(2): a, c, g, or t
  modified_base (8)..(10): a, c, g, or t
  modified_base (13)..(16): a, c, g, or t
  modified_base (19)..(21): a, c, g, or t
  modified_base (23)..(24): a, c, g, or t
nntttcgnnnttnnnntgnnncnntttcgcgtttttcgtgcgccgcttca

SEQ ID NO: 70 - DNA, 50 bp, Artificial Sequence
  source: /note=“Description of Artificial Sequence Synthetic oligonucleotide”
  modified_base (8)..(10): a, c, g, or t
  modified_base (13)..(16): a, c, g, or t
  modified_base (19)..(21): a, c, g, or t
  modified_base (23)..(24): a, c, g, or t
  modified_base (30)..(32): a, c, g, or t
  modified_base (35)..(38): a, c, g, or t
  modified_base (41)..(43): a, c, g, or t
  modified_base (45)..(49): a, c, g, or t
gttttcgnnnttnnnntgnnncnntttcgnnnttnnnntgnnncnnnnna

SEQ ID NO: 71 - DNA, 50 bp, Artificial Sequence
  source: /note=“Description of Artificial Sequence Synthetic oligonucleotide”
  modified_base (13)..(16): a, c, g, or t
  modified_base (19)..(21): a, c, g, or t
  modified_base (23)..(24): a, c, g, or t
  modified_base (30)..(32): a, c, g, or t
  modified_base (35)..(38): a, c, g, or t
  modified_base (41)..(43): a, c, g, or t
  modified_base (45)..(49): a, c, g, or t
gttttcgcatttnnnntgnnncnntttcgnnnttnnnntgnnncnnnnna

SEQ ID NO: 72 - DNA, 50 bp, Artificial Sequence
  source: /note=“Description of Artificial Sequence Synthetic oligonucleotide”
  modified_base (19)..(21): a, c, g, or t
  modified_base (23)..(24): a, c, g, or t
  modified_base (30)..(32): a, c, g, or t
  modified_base (35)..(38): a, c, g, or t
  modified_base (41)..(43): a, c, g, or t
  modified_base (45)..(49): a, c, g, or t
gttttcgcatttatcgtgnnncnntttcgnnnttnnnntgnnncnnnnna

SEQ ID NO: 73 - DNA, 50 bp, Artificial Sequence
  source: /note=“Description of Artificial Sequence Synthetic oligonucleotide”
  modified_base (23)..(24): a, c, g, or t
  modified_base (30)..(32): a, c, g, or t
  modified_base (35)..(38): a, c, g, or t
  modified_base (41)..(43): a, c, g, or t
  modified_base (45)..(49): a, c, g, or t
gttttcgcatttatcgtgaaacnntttcgnnnttnnnntgnnncnnnnna

SEQ ID NO: 74 - DNA, 50 bp, Artificial Sequence
  source: /note=“Description of Artificial Sequence Synthetic oligonucleotide”
  modified_base (30)..(32): a, c, g, or t
  modified_base (35)..(38): a, c, g, or t
  modified_base (41)..(43): a, c, g, or t
  modified_base (45)..(49): a, c, g, or t
gttttcgcatttatcgtgaaacgctttcgnnnttnnnntgnnncnnnnna

SEQ ID NO: 75 - DNA, 50 bp, Artificial Sequence
  source: /note=“Description of Artificial Sequence Synthetic oligonucleotide”
  modified_base (41)..(43): a, c, g, or t
  modified_base (45)..(49): a, c, g, or t
gttttcgcatttatcgtgaaacgctttcgcgtttttcgtgnnncnnnnna

SEQ ID NO: 76 - DNA, 50 bp, Artificial Sequence
  source: /note=“Description of Artificial Sequence Synthetic oligonucleotide”
  modified_base (8)..(10): a, c, g, or t
  modified_base (13)..(16): a, c, g, or t
  modified_base (19)..(21): a, c, g, or t
  modified_base (23)..(24): a, c, g, or t
gttttcgnnnttnnnntgnnncnntttcgcgtttttcgtgcgccgcttca

SEQ ID NO: 77 - DNA, 50 bp, Artificial Sequence
  source: /note=“Description of Artificial Sequence Synthetic oligonucleotide”
  modified_base (13)..(16): a, c, g, or t
  modified_base (19)..(21): a, c, g, or t
  modified_base (23)..(24): a, c, g, or t
gttttcgcatttnnnntgnnncnntttcgcgtttttcgtgcgccgcttca

SEQ ID NO: 78 - DNA, 20 bp, Artificial Sequence
  source: /note=“Description of Artificial Sequence Synthetic primer”
cccacatccgctctaaccga

SEQ ID NO: 79 - DNA, 21 bp, Artificial Sequence
  source: /note=“Description of Artificial Sequence Synthetic primer”
ccccgcataaacacctctctt

SEQ ID NO: 80 - DNA, 67 bp, Artificial Sequence
  source: /note=“Description of Artificial Sequence Synthetic primer”
aatgatacggcgaccaccgagatctacactatagcctatgcgacactcgtgaaacgctttcgcgttt

SEQ ID NO: 81 - DNA, 67 bp, Artificial Sequence
  source: /note=“Description of Artificial Sequence Synthetic primer”
aatgatacggcgaccaccgagatctacacatagaggcatgcgacactcgtgaaacgctttcgcgttt

SEQ ID NO: 82 - DNA, 67 bp, Artificial Sequence
  source: /note=“Description of Artificial Sequence Synthetic primer”
aatgatacggcgaccaccgagatctacaccctatcctatgcgacactcgtgaaacgctttcgcgttt

SEQ ID NO: 83 - DNA, 64 bp, Artificial Sequence
  source: /note=“Description of Artificial Sequence Synthetic primer”
caagcagaagacggcatacgagatattactcgcgaggtcgagtgcatgaaacgctttcgcgttt

SEQ ID NO: 84 - DNA, 64 bp, Artificial Sequence
  source: /note=“Description of Artificial Sequence Synthetic primer”
caagcagaagacggcatacgagattccggagacgaggtcgagtgcatgaaacgctttcgcgttt

SEQ ID NO: 85 - DNA, 64 bp, Artificial Sequence
  source: /note=“Description of Artificial Sequence Synthetic primer”
caagcagaagacggcatacgagatcgctcattcgaggtcgagtgcatgaaacgctttcgcgttt

SEQ ID NO: 86 - DNA, 28 bp, Artificial Sequence
  source: /note=“Description of Artificial Sequence Synthetic primer”
atgcgacactcgttcgtgcgtcagttca

SEQ ID NO: 87 - DNA, 31 bp, Artificial Sequence
  source: /note=“Description of Artificial Sequence Synthetic primer”
cgaggtcgagtgcagttcgtgcgtcagttca

SEQ ID NO: 88 - DNA, 31 bp, Artificial Sequence
  source: /note=“Description of Artificial Sequence Synthetic primer”
tgaactgacgcacgaactgcactcgacctcg

SEQ ID NO: 89 - DNA, 27 bp, Unknown
  source: /note=“Description of Unknown R1 sequence of wild type Mu transposon end sequence”
ctttcgcgtttttcgtgcgccgcttca

SEQ ID NO: 90 - DNA, 23 bp, Unknown
  source: /note=“Description of Unknown R2 sequence of wild type Mu transposon end sequence”
gttttcgcatttatcgtgaaacg

SEQ ID NO: 91 - DNA, 20 bp, Artificial Sequence
  source: /note=“Description of Artificial Sequence Synthetic primer”
cccacatccgctctaaccga

SEQ ID NO: 92 - DNA, 21 bp, Artificial Sequence
  source: /note=“Description of Artificial Sequence Synthetic primer”
ccccgcataaacacctctctt

SEQ ID NO: 93 - DNA, 19 bp, Artificial Sequence
  source: /note=“Description of Artificial Sequence Synthetic primer”
gcgagtgacgcttggtgaa

SEQ ID NO: 94 - DNA, 21 bp, Artificial Sequence
  source: /note=“Description of Artificial Sequence Synthetic primer”
ggaaccaggggtaggtgatga

SEQ ID NO: 95 - DNA, 20 bp, Artificial Sequence
  source: /note=“Description of Artificial Sequence Synthetic primer”
cagccagtgcttgttgcttg

SEQ ID NO: 96 - DNA, 20 bp, Artificial Sequence
  source: /note=“Description of Artificial Sequence Synthetic primer”
ccctagacagggagtgcgat

SEQ ID NO: 97 - DNA, 22 bp, Artificial Sequence
  source: /note=“Description of Artificial Sequence Synthetic primer”
acaaatttctaccctctcacga

SEQ ID NO: 98 - DNA, 20 bp, Artificial Sequence
  source: /note=“Description of Artificial Sequence Synthetic primer”
ctttgagagcctttagccgc

SEQ ID NO: 99 - DNA, 20 bp, Artificial Sequence
  source: /note=“Description of Artificial Sequence Synthetic primer”
ccagtgcccactcaagtcat

SEQ ID NO: 100 - DNA, 20 bp, Artificial Sequence
  source: /note=“Description of Artificial Sequence Synthetic primer”
aggtggacatcgatgagtgc

SEQ ID NO: 101 - DNA, 20 bp, Artificial Sequence
  source: /note=“Description of Artificial Sequence Synthetic primer”
ggtgtctagctgtcagtggt

SEQ ID NO: 102 - DNA, 20 bp, Artificial Sequence
  source: /note=“Description of Artificial Sequence Synthetic primer”
tgtcgttcacacagccagaa

SEQ ID NO: 103 - DNA, 50 bp, Artificial Sequence
  source: /note=“Description of Artificial Sequence Synthetic oligonucleotide”
gttttcgcatttaatatgactcactatagcgtttttcgtgcgccgcttca

SEQ ID NO: 104 - DNA, 54 bp, Artificial Sequence
  source: /note=“Description of Artificial Sequence Synthetic oligonucleotide”
ctagtgaagcggcgcacgaaaaacgctatagtgagtcatattaaatgcgaaaac

SEQ ID NO: 105 - DNA, 50 bp, Artificial Sequence
  source: /note=“Description of Artificial Sequence Synthetic oligonucleotide”
gttttcgcatttaatatgactcactttagcgtttttcgtgcgccgcttca

SEQ ID NO: 106 - DNA, 54 bp, Artificial Sequence
  source: /note=“Description of Artificial Sequence Synthetic oligonucleotide”
ctagtgaagcggcgcacgaaaaacgctaaagtgagtcatattaaatgcgaaaac

SEQ ID NO: 107 - DNA, 50 bp, Artificial Sequence
  source: /note=“Description of Artificial Sequence Synthetic oligonucleotide”
gttttcgcatttatcgtgaaacgctttcgcgtattacgactcactataga

SEQ ID NO: 108 - DNA, 54 bp, Artificial Sequence
  source: /note=“Description of Artificial Sequence Synthetic oligonucleotide”
ctagtctatagtgagtcgtaatacgcgaaagcgtttcacgataaatgcgaaaac

SEQ ID NO: 109 - DNA, 50 bp, Artificial Sequence
  source: /note=“Description of Artificial Sequence Synthetic oligonucleotide”
gttttcgcatttatcgtgaaacgctttcgcgtattacgtctcactataga

SEQ ID NO: 110 - DNA, 54 bp, Artificial Sequence
  source: /note=“Description of Artificial Sequence Synthetic oligonucleotide”
ctagtctatagtgagacgtaatacgcgaaagcgtttcacgataaatgcgaaaac

SEQ ID NO: 111 - DNA, 50 bp, Artificial Sequence
  source: /note=“Description of Artificial Sequence Synthetic oligonucleotide”
gttttcgcatttatcgtgaaacgctttcgcgttttacgtctcactataga

SEQ ID NO: 112 - DNA, 54 bp, Artificial Sequence
  source: /note=“Description of Artificial Sequence Synthetic oligonucleotide”
ctagtctatagtgagacgtaaaacgcgaaagcgtttcacgataaatgcgaaaac

SEQ ID NO: 113 - DNA, 50 bp, Artificial Sequence
  source: /note=“Description of Artificial Sequence Synthetic oligonucleotide”
tatttcgactcactattgaaacgctttcgcgtttttcgtgcgccgcttca

SEQ ID NO: 114 - DNA, 54 bp, Artificial Sequence
  source: /note=“Description of Artificial Sequence Synthetic oligonucleotide”
ctagtgaagcggcgcacgaaaaacgcgaaagcgtttcaatagtgagtcgaaata

(19) United States
(12) Patent Application Publication
(10) Pub. No.: US 2022/0396788 A1
(43) Pub. Date: Dec. 15, 2022

(54) RECOMBINANT TRANSPOSON ENDS

(71) Applicant: THERMO FISHER SCIENTIFIC BALTICS, UAB, Vilnius (LT)

(72) Inventors: Arvydas LUBYS, Vilnius (LT); Paulius MIELINIS, Palanga (LT); Linas ZAKRYS, Vilnius (LT); Rasa SUKACKAITE, Vilnius (LT)

(21) Appl. No.: 17/642,849

(22) PCT Filed: Sep. 14, 2020
(86) PCT No.: PCT/EP2020/075663

§ 371 Date: Mar. 14, 2022

Related U.S. Application Data
(60) Provisional application No. 62/899,468, filed on Sep. 12, 2019.
(60) Provisional application No. 63/058,939, filed on Jul. 30, 2020.

(51) Int. Cl. C12N 15/10; C12Q 1/6806

(52) U.S. Cl. CPC C12N 15/1065; C12Q 1/6806

(57) ABSTRACT

Recombinant transposon end nucleic acids are described that can incorporate barcodes, sequencing primers, or other functional biological sequences. This application also describes mixtures and uses of the recombinant transposon end nucleic acids.

### SEQUENCE LISTING

The instant application contains a Sequence Listing which has been submitted electronically in ASCII format and is hereby incorporated by reference in its entirety. Said ASCII copy, created on Sep. 14, 2020, is named LT01488PCT_SL_1.txt and is 39,818 bytes in size.

### FIELD

This application relates to recombinant transposon end nucleic acids that can incorporate barcodes, sequencing primers, or other functional biological sequences into known or unknown nucleic acids in a sample. This application also relates to mixtures and uses of the recombinant transposon end nucleic acids.

### BACKGROUND

Next generation sequencing is a powerful tool to investigate a genome with an ease. Sequencing library construction begins with an adapter addition, regardless of the sequencing system. Adapters are introduced by using various DNA library preparation methods, such as ligation-based or tagmentation-based methods. Ligation-based methods use pre-fragmented DNA and ligate adapters in a random fashion, while tagmentation-based methods rely on simultaneous random fragmentation of DNA by a transposase and insertion of a transposon sequence in both ends of the resulting DNA fragment. The inserted transposon sequence can then be used as a basis for adapter sequence and/or sequencing primer binding site. Tn5 and MuA are the two commonly used transposase/transposon systems.

Current technological advances in sample preparation and next-generation sequencing field allow sequencing of individual cells. In order to identify and sort data of single cells and each of their nucleic acids after sequencing and to eliminate sequencing noise, unique barcodes (such as unique molecular identifiers, UMIs) have to be used (See, e.g. Islam et al., Nature Methods 11:163-166 (2014)). In the case of tagmentation, such unique sequences are introduced by adding tag sequences outside the transposon end (in case of transposon ends used by Tn5 transposase). Methods are evolving that require rather long stretches of identification or unique labeling sequences, such as 12-16 nucleotide (nt) length UMIs. For example, in application such as LIANTI (Linear Amplification via Transposon Insertion), a sequence of T7 promoter is introduced in the proximity of the transposon end from Tn5 transposase-based system, which in result is capable of generating copies of a genome in a linear pre-amplification reaction, together with the sequencing primer binding site and a barcode (Chen et al., Science 356(6334): 189-194 (2017)). This rather long stretch of sequence is provided in the form of a tag that is additionally provided next to the transposon end (the 19 bp double stranded transposase binding site) sequence. When coupling barcoding or other sequence introduction with Tn5 transposase-based system, modifications may be introduced outside the Tn5 transposon mosaic end (ME) sequence, thus generating an additional transposon sequence in the final sequencing-ready molecule.

Thus, a transposase-based system is required that would have a minimal length of sequence between the binding site of sequencing primer and the sequence to be sequenced, and at the same time could add the required barcodes and other identifiers, including longer sequences.

### SUMMARY

This application describes means to alter Mu transposon end sequences to introduce a sequence of interest. In some embodiments, the introduced sequence is a random sequence. In some embodiments, the introduced sequence is a specific sequence, such as a unique barcode, primer binding site, or functional biological sequence. This application describes alterations that can be made in the R1 and/or R2 regions of the Mu transposon end structure.

In some embodiments, a composition comprises a mixture of at least 25 different recombinant transposon end nucleic acids each independently comprising the nucleotide sequence of 5′-NNTTTCGNNNTTNNNNTGNNNCNNTTTCGNNNTTNNNNTGNNNCNNNNNA-3′ (SEQ ID NO: 20); wherein in each nucleic acid each N is independently chosen from A, C, G, and T.

In some embodiments, a composition comprises a mixture of recombinant transposon end nucleic acids comprising the nucleotide sequence of 5′-NNTTTCGNNNTTNNNNTGNNNCNNTTTCG CGTTTNNNNTGNNNCNNNNNA-3′ (SEQ ID NO: 66); wherein in each nucleic acid each N is independently chosen from A, C, G, and T.

In some embodiments, a composition comprises a mixture of recombinant transposon end nucleic acids comprising the nucleotide sequence of 5′-NNTTTCGNNNTTNNNNTGNNNCNNTTTCGCGT TTTTCGTGNNNCNNNNNA-3′ (SEQ ID NO: 67); wherein in each nucleic acid each N is independently chosen from A, C, G, and T.

In some embodiments, a composition comprises a mixture of recombinant transposon end nucleic acids comprising the nucleotide sequence of 5′-NNTTTCGNNNTTNNNNTGNNNCNNTTTCG CGTTTTTCGTGCGCCNNNNNA-3′ (SEQ ID NO: 68); wherein in each nucleic acid each N is independently chosen from A, C, G, and T.

In some embodiments, a composition comprises a mixture of recombinant transposon end nucleic acids comprising the nucleotide sequence of 5′-NNTTTCGNNNTTNNNNTGNNNCNNTTTCGCGTTTTTC GTGCGCCGCTTCA-3′ (SEQ ID NO: 69); wherein in each nucleic acid each N is independently chosen from A, C, G, and T.

In some embodiments, a composition comprises a mixture of recombinant transposon end nucleic acids comprising the nucleotide sequence of 5′-GTTTTCGCATTTATCGTGAAACGCTTTCGNNNTTNNNNTGNNN CNNNNNA-3′ (SEQ ID NO: 74); wherein in each nucleic acid each N is independently chosen from A, C, G, and T.

In some embodiments, a composition comprises a mixture of recombinant transposon end nucleic acids comprising the nucleotide sequence of 5′-GTTTTCGCATTTATCGTGAAACGCTTTCGCGTTTNNNNTGNNN CNNNNNA-3′ (SEQ ID NO: 16); wherein in each nucleic acid each N is independently chosen from A, C, G, and T.

In some embodiments, a composition comprises a mixture of recombinant transposon end nucleic acids comprising the nucleotide sequence of 5′-GTTTTCGCATTTATCGTGAAACGCTTTCGCGTTTTTCGTGNNN CNNNNNA-3′ (SEQ ID NO: 75); wherein in each nucleic acid each N is independently chosen from A, C, G, and T.

In some embodiments, a composition comprises a mixture of recombinant transposon end nucleic acids comprising the nucleotide sequence of 5′-GTTTTCGCATTTATCGTGAAACGCTTTCGCGTTTTTCGTG CGCCNNNNNA-3′ (SEQ ID NO: 12); wherein in each nucleic acid each N is independently chosen from A, C, G, and T.

In some embodiments, at least one transposon end nucleic acid of a composition comprising of the mixture of recombinant transposon end nucleic acids has a sequence that has a nucleotide substitution at one or more positions corresponding to positions selected from 1, 2, 8, 9, 10, 13, 14, 15, 16, 19, 20, 21, 23, 24, 37, 41, or 49 positions of SEQ ID NO: 1.

In some embodiments, each nucleic acid in a compositions comprising the mixture of recombinant transposon end nucleic acids is unique.

In some embodiments, a composition comprises a mixture of recombinant transposon end nucleic acids comprising at least 25, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000, 11000, 12000, 13000, 14000, 15000, 16000, 17000, 18000, 19000, 20000, 25000, 50000, 75000, 100000, 200000, 300000, 400000, 500000, 600000, 700000, 800000, 900000, 1000000, or more transposon end nucleic acids.

In some embodiments, a composition comprises at least one transposase and a mixture of recombinant transposon end nucleic acids.

In some embodiments, a method of fragmenting a sample comprising nucleic acids, comprising contacting the sample with a composition comprising at least one transposase and a mixture of recombinant transposon end nucleic acids is provided.

In some embodiments, a sample is obtained from one cell.

In some embodiments, a method of generating a population of uniquely barcoded nucleic acid fragments from a sample comprising nucleic acids is provided, comprising contacting the sample with a composition comprising at least one transposase and a mixture of recombinant transposon end nucleic acids, wherein the composition comprises at least 25, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000, 11000, 12000, 13000, 14000, 15000, 16000, 17000, 18000, 19000, 20000, 25000, 50000, 75000, 100000, 200000, 300000, 400000, 500000, 600000, 700000, 800000, 900000, 1000000, or more transposon end nucleic acids with different sequences.

In some embodiments, a method of generating a population of barcoded nucleic acid fragments from a sample comprising nucleic acids comprises contacting the sample with a composition comprising at least one transposase and a mixture of recombinant transposon end nucleic acids, wherein transposon end nucleic acids barcode the nucleic acid fragments from the sample.

In some embodiments, a method of fragmenting a sample comprising nucleic acids or a method of generating a population of barcoded nucleic acid fragments from a sample comprising nucleic acids, further comprises sequencing the population of barcoded nucleic acid fragments, that can be followed by any of sequence assembly, mutation analysis, allele analysis, copy number analysis, and/or haplotype analysis. In some embodiments, sequences of the barcodes are used for realignment of sequences in haplotype analysis. In some embodiments, sequences of the barcodes are used to identify unique fragments generated during fragmentation of the sample. In some embodiments, the sequences of the barcodes are used to identify unique fragments generated during fragmentation of the sample

In some embodiments, a recombinant transposon end nucleic acid comprises a variant of the nucleotide sequence of SEQ ID NO: 1 having:
a. nucleotide substitutions at one or more positions corresponding to positions selected from 1, 2, 8, 9, 10, 13, 14, 15, 16, 19, 20, 21, 23, 24, 37, 41, or 49 positions of SEQ ID NO: 1;
b. nucleotide substitution at positions 6, 11, 12, 17, 18, 22, 25, 26 and/or 28, and, optionally, one or more nucleotide substitutions at positions corresponding to N positions in SEQ ID NO: 76;
c. nucleotide substitution at positions 33, 39, 40, and/or 44, and, optionally, one or more nucleotide substitutions at positions corresponding to N positions in SEQ ID NO: 73;
d. nucleotide substitution at positions 11 and 12, and, optionally, one or more nucleotide substitutions at positions corresponding to N positions in SEQ ID NO: 76;
e. nucleotide substitutions at positions 6, 12, and 17, and, optionally, one or more nucleotide substitutions at positions corresponding to N positions in SEQ ID NO: 76;
f. nucleotide substitutions at positions 12, 18, 22, and 25, and, optionally, one or more nucleotide substitutions at positions corresponding to N positions in SEQ ID NO: 76;
g. nucleotide substitutions at positions 40, and, optionally, one or more nucleotide substitutions at positions corresponding to N positions in SEQ ID NO: 74;
h. nucleotide substitutions at positions 33 and 40, and, optionally, one or more nucleotide substitutions at positions corresponding to N positions in SEQ ID NO: 74;
i. nucleotide substitutions at positions 39, 40, and 44, and, optionally, one or more nucleotide substitutions at positions corresponding to N positions in SEQ ID NO: 74;
j. nucleotide substitutions at positions 33, 39, and 40, and, optionally, one or more nucleotide substitutions at positions corresponding to N positions in SEQ ID NO: 74;
k. nucleotide substitution at position 28, and, optionally, one or more nucleotide substitutions at positions corresponding to N positions in SEQ ID NO: 77;
l. nucleotide substitutions at positions 26, and 28, and, optionally, one or more nucleotide substitutions at positions corresponding to N positions in SEQ ID NO: 77;
m. nucleotide substitutions at positions 17, 26, and 28, and, optionally, one or more nucleotide substitutions at positions corresponding to N positions in SEQ ID NO: 77;
n. nucleotide substitutions at positions 33, 34, 39, and 40, and, optionally, one or more nucleotide substitutions at positions corresponding to N positions in SEQ ID NO: 16; or
o. nucleotide substitutions of any one of (a)-(n) above and further comprising one, two, three, four, or five additional nucleotide substitutions compared to the nucleotide sequence of SEQ ID NO: 1.

In some embodiments, a recombinant transposon end nucleic acid nucleotide substitutions generate an additional biological function in the recombinant transposon end nucleic acid. In some embodiments, the additional biological function comprises (i) a primer binding site; (ii) all or part of a restriction endonuclease recognition site; and/or (iii) all or part of a promoter sequence. In some embodiments, the additional biological function is a promoter sequence. In some embodiments, the promoter sequence is a T3 or T7 promoter. In some embodiments, a recombinant transposon end nucleic acid nucleotide substitutions further generate one or more barcodes.

In some embodiments, a composition comprising one or more transposase and the recombinant transposon end nucleic acid with one or more nucleotide substitutions is provided. In some embodiments, a composition further comprises one or more additional recombinant transposon end nucleic acid, wherein the recombinant transposon end nucleic acids have different nucleotide sequences. In some embodiments, a method of generating a population of nucleic acid fragments from a sample comprising nucleic acids comprises contacting the sample with one or more composition.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides a transposon end sequence and its non-conserved regions. Transposon end DNA (comprised of SEQ ID NO: 1 and SEQ ID NO: 2) is composed of two MuA transposase binding elements, R1 (SEQ ID NO: 89) and R2 (SEQ ID NO: 90). The regions that do not interact with protein domains (boxed in the figure) provide structural function. The very 3′ adenosine nucleotide is required for cleavage.

FIG. 2 shows synthesis of a transposon end with randomized regions. A primer complementary to a transposon end template harboring randomized regions within non-conserved regions is annealed and extended using a DNA polymerase resulting in double-stranded 70 nucleotide pre-transposon end fragment that is cut at the 3′ transposon end's A nucleotide by an endonuclease leaving a functional transposon end with protruding 5′ end at the non-transferred strand. Non-conserved sites, boxed, are shown here substituted as N's. The extension primer is shown as an arrow. The striped box represents a restriction endonuclease cutting site.

FIG. 3 shows the structure of pre-transposon and transposon ends. Non-conserved sites, boxed, are shown here substituted as shaded N's. Conserved sequences are shown in bold.

FIG. 4 shows EMSA analysis of MuA transposomes comprising random sequences. Analysis was carried out on 2% agarose gel containing 0.5 μg/mL Ethidium bromide and 87 μg/mL BSA and heparin. 5 μL of each loaded sample contains 2 μL of each transposome complex, 1 μL 6× TriTrack DNA Loading Dye (Thermo Scientific, Cat. No. R1161) and 2 μL of water. GeneRuler Low Range DNA Ladder (Thermo Scientific, Cat. No. SM1193) was used.

FIGS. 5A-5D show transposome activity evaluation. 100 ng of Escherichia virus lambda gDNA was fragmented for 5 minutes using 1.5 μL of each transposome complex, following by an SDS addition to a final concentration of 0.4% to stop the reaction. Reaction products were purified using GeneJET NGS Cleanup Kit, protocol A (Thermo Scientific, Cat. No. K0851). Reaction products were analyzed on Agilent High Sensitivity DNA Kit (Agilent, Cat. No. 5067-4626). N0 (FIG. 5A), N5 (FIG. 5B), N12 (FIG. 5C), and N29 (FIG. 5D) randomized nucleotide carrying transposome complexes were used.

FIG. 6 shows barcode unique molecular identifier (UMI, also known as barcodes) utility in tagmentation-mediated DNA library construction. In this embodiment, the barcode is a molecular barcode (i.e., a UMI) Unique sequences carrying transposon ends are inserted during tagmentation. In the case of two or more similar sequences present in DNA library, a barcode/UMI acts as an identifier of whether a sequence is a PCR duplicate or an original two copies of molecules.

FIG. 7 provides sequences of representative transposon ends containing unique barcodes. Underlined nucleotides indicate 4 base pair unique transposon end identifiers. Tetranucleotides in this specific Figure were chosen by a rule that sequences have to differ by at least 2 nucleotides across all tetramers. The sequences provided in this figure comprise SEQ ID NOs: 1-2 and 22-45.

FIG. 8 provides EMSA analysis of MuA transposomes that all contain individual unique sequences. Analysis was carried out on 2% agarose gel containing 0.5 μg/mL Ethidium bromide and 87 μg/mL BSA and heparin. 5 μL of each loaded sample contains 2 μL of each transposome complex, 1 μL 6× TriTrack DNA Loading Dye (Thermo Scientific, Cat. No. R1161), and 2 μL of water. GeneRuler Low Range DNA Ladder (Thermo Scientific, Cat. No. SM1193) marker was used.

FIGS. 9A-9N shows transposome activity evaluation. 100 ng of Escherichia virus lambda gDNA was fragmented for 5 minutes using 1.5 μL of each transposome complex, following by an SDS addition to a final concentration of 0.4% to stop the reaction. Reaction products were purified using GeneJET NGS Cleanup Kit, protocol A (Thermo Scientific, Cat. No. K0851). Reaction products were analyzed on Agilent High Sensitivity DNA Kit (Agilent, Cat. No. 5067-4626). Twelve unique transposome complexes and two controls were used.

FIG. 10 shows unique transposon end identifier sequence (UTI) utility in haplotype assembly. UTIs comprising recombinant transposon end pairs are inserted during tagmentation. The cleaved DNA ends both have the same unique sequence (i.e., a barcode); therefore, reads can be re-aligned using these tag sequences after being sequenced.

FIG. 11 shows sequences of oligonucleotides wherein a custom primer binding site has been introduced into a Mu transposon end.

FIG. 12 shows EMSA analysis of MuA transposomes containing custom primer binding sites. Analysis was carried out on 2% agarose gel containing 0.5 μg/mL Ethidium bromide and 87 μg/mL BSA and heparin. 5 μL of each loaded sample contains 2 μL of each transposome complex, 1 μL 6× TriTrack DNA Loading Dye (Thermo Scientific, Cat. No. R1161) and 2 μL of water. GeneRuler Low Range DNA Ladder (Thermo Scientific, Cat. No. SM1193) marker was used.

FIGS. 13A-13C shows transposome activity evaluation. 100 ng of Escherichia virus lambda gDNA was fragmented for 5 minutes using 1.5 μL of each transposome complex, following by an SDS addition to a final concentration of 0.4% to stop the reaction. Reaction products were purified using GeneJET NGS Cleanup Kit, protocol A (Thermo Scientific, Cat. No. K0851). Reaction products were analyzed on Agilent High Sensitivity DNA Kit (Agilent, Cat. No. 5067-4626). Tn-SEQ1 (FIG. 13A), Tn-SEQ2.1 (FIG. 13B), and Tn-SEQ2.2 (FIG. 13 C) transposon end containing complexes were used.

FIG. 14 shows functional biological sequences introduced into a Mu transposon end. The boxed sequences correspond to a T3 promoter (SEQ ID NO: 54) or T7 promoter sequence (SEQ ID NO: 55).

FIG. 15 shows use of transposon ends containing UMIs for detection of rare mutations. Target DNA molecules (black boxes) are fragmented and tagged with UMIs during tagmentation. UMIs with different sequences are marked as boxes with different pattern.

FIGS. 16A-16F. Low rate mutation detection using the tagmentation with transposon ends with UMIs approach. FIG. 16A-16B—the wild-type plasmid was spiked with the double mutant (A940G, T3428G) plasmid at quantitative ratios of 1:200 and 1:1000, and then subjected to MuA-UMI tagmentation and sequencing. Variant fractions, defined as a ratio between confident variants and all confident clusters (reads), are plotted against the 3.75 kbp region of interest. FIG. 16C-16D—variant fractions plotted against the target region when the target region was preamplified from wild-type/mutant plasmid mixtures with Taq DNA polymerase prior to MuA-UMI tagmentation. FIG. 16E-16F—variant fractions plotted against the target region when the target region was preamplified from wild-type/mutant plasmid mixtures with Platinum SuperFi II DNA polymerase prior to tagmentation. True mutations indicated by arrows, where available.

### DESCRIPTION OF THE SEQUENCES

A listing of certain sequences referenced herein is provided.

Description of the Sequences and SEQ ID Nos

| Description | Sequence | # |
|---|---|---|
| Wild type Mu transposon end | GTTTTCGCATTTATCGTGAAACGCTTTCGCGTTTTTCGTG | 1 |
| sequence, transferred strand | CGCCGCTTCA | |
| Wild type Mu transposon end | CTAGTGAAGCGGCGCACGAAAAACGCGAAAGCGTTTCACG | 2 |
| sequence, non-transferred | ATAAATGCGAAAAC | |
| strand | | |
| Template oligo for control (N0) | GTTTTCGCATTTATCGTGAAACGCTTTCGCGTTTTTCGTG | 3 |
| transposon synthesis | CGCCGCTTCACTAGTTTGGCGTAATCGCCG | |
| Template oligo for control (N5) | GTTTTCGCATTTATCGTGAAACGCTTTCGCGTTTTTCGTG | 4 |
| transposon synthesis | CGCCNNNNNACTAGTTTGGCGTAATCGCCG | |
| Template oligo for control | GTTTTCGCATTTATCGTGAAACGCTTTCGCGTTTNNNNTG | 5 |
| (N12) transposon synthesis | NNNCNNNNNACTAGTTTGGCGTAATCGCCG | |
| Template oligo for control | NNTTTCGNNNTTNNNNTGNNNCNNTTTCGNNNTTNNNNTG | 6 |
| (N29) transposon synthesis | NNNCNNNNNACTAGTTTGGCGTAATCGCCG | |
| Extension oligo for degenerate | CGGCGATTACGCCAAACTAGTG | 7 |
| transposon synthesis | | |
| Control pre-transposon end | GTTTTCGCATTTATCGTGAAACGCTTTCGCGTTTTTCGTG | 8 |
| containing no randomized | CGCCGCTTCACTAGTTTGGCGTAATCGCCG | |
| nucleotides, transferred strand | | |
| Control pre-transposon end | CGGCGATTACGCCAAACTAGTGAAGCGGCGCACGAAAAAC | 9 |
| containing no randomized | GCGAAAGCGTTTCACGATAAATGCGAAAAC | |
| nucleotides, non-transferred | | |
| strand | | |
| Pre-transposon end containing 5 | GTTTTCGCATTTATCGTGAAACGCTTTCGCGTTTTTCGTG | 10 |
| randomized nucleotides, | CGCCNNNNNACTAGTTTGGCGTAATCGCCG | |
| transferred strand | | |
| Pre-transposon end containing 5 | CGGCGATTACGCCAAACTAGTNNNNNGGCGCACGAAAAAC | 11 |
| randomized nucleotides, non- | GCGAAAGCGTTTCACGATAAATGCGAAAAC | |
| transferred strand | | |
| Transposon end containing 5 | GTTTTCGCATTTATCGTGAAACGCTTTCGCGTTTTTCGTG | 12 |
| randomized nucleotides, top | CGCCNNNNNA | |
| strand | | |
| Transposon end containing 5 | CTAGTNNNNNGGCGCACGAAAAACGCGAAAGCGTTTCACG | 13 |
| randomized nucleotides, non- | ATAAATGCGAAAAC | |
| transferred strand | | |
| Pre-transposon end containing | GTTTTCGCATTTATCGTGAAACGCTTTCGCGTTTNNNNTG | 14 |
| 12 randomized nucleotides, | NNNCNNNNNACTAGTTTGGCGTAATCGCCG | |
| transferred strand | | |
| Pre-transposon end containing | CGGCGATTACGCCAAACTAGTNNNNNGNNNCANNNNAAAC | 15 |
| 12 randomized nucleotides, non- | GCGAAAGCGTTTCACGATAAATGCGAAAAC | |
| transferred strand | | |
| Transposon end containing 12 | GTTTTCGCATTTATCGTGAAACGCTTTCGCGTTTNNNNTG | 16 |
| randomized nucleotides, | NNNCNNNNNA | |
| transferred strand | | |
| Transposon end containing 12 | CTAGTNNNNNGNNNCANNNNAAACGCGAAAGCGTTTCACG | 17 |
| randomized nucleotides, non- | ATAAATGCGAAAAC | |
| transferred strand | | |
| Pre-transposon end containing | NNTTTCGNNNTTNNNNTGNNNCNNTTTCGNNNTTNNNNTG | 18 |
| 29 randomized nucleotides, | NNNCNNNNNACTAGTTTGGCGTAATCGCCG | |
| transferred strand | | |
| Pre-transposon end containing | CGGCGATTACGCCAAACTAGTNNNNNGNNNCANNNNAANN | 19 |
| 29 randomized nucleotides, non- | NCGAAANNGNNNCANNNNAANNNCGAAANN | |
| transferred strand | | |
| Transposon end containing 29 | NNTTTCGNNNTTNNNNTGNNNCNNTTTCGNNNTTNNNNTG | 20 |
| randomized nucleotides, | NNNCNNNNNA | |
| transferred strand | | |
| Transposon end containing 29 | CTAGTNNNNNGNNNCANNNNAANNNCGAAANNGNNNCANN | 21 |
| randomized nucleotides, non- | NNAANNNCGAAANN | |
| transferred strand | | |
| Transposon end containing | GTTTTCGCATTTATCGTGAAACGCTTTCGCGTTTTTCGTG | 22 |
| internal TACG barcode, | CGCCTACGCA | |
| transferred strand | | |
| Transposon end containing | CTAGTGCGTAGGCGCACGAAAAACGCGAAAGCGTTTCACG | 23 |
| internal TACG barcode, non- | ATAAATGCGAAAAC | |
| transferred strand | | |
| Transposon end containing | GTTTTCGCATTTATCGTGAAACGCTTTCGCGTTTTTCGTG | 24 |
| internal GTAC barcode, | CGCCGTACCA | |
| transferred strand | | |
| Transposon end containing | CTAGTGGTACGGCGCACGAAAAACGCGAAAGCGTTTCACG | 25 |
| internal GTAC barcode, non- | ATAAATGCGAAAAC | |
| transferred strand | | |
| Transposon end containing | GTTTTCGCATTTATCGTGAAACGCTTTCGCGTTTTTCGTG | 26 |
| internal CGCA barcode, | CGCCCGCACA | |
| transferred strand | | |
| Transposon end containing | CTAGTGTGCGGGCGCACGAAAAACGCGAAAGCGTTTCACG | 27 |
| internal CGCA barcode, non- | ATAAATGCGAAAAC | |
| transferred strand | | |
| Transposon end containing | GTTTTCGCATTTATCGTGAAACGCTTTCGCGTTTTTCGTG | 28 |
| internal ACATbarcode, | CGCCACATCA | |
| transferred strand | | |
| Transposon end containing | CTAGTGATGTGGCGCACGAAAAACGCGAAAGCGTTTCACG | 29 |
| internal ACATbarcode, non- | ATAAATGCGAAAAC | |
| transferred strand | | |
| Transposon end containing | GTTTTCGCATTTATCGTGAAACGCTTTCGCGTTTTTCGTG | 30 |
| internal CATG barcode, | CGCCCATGCA | |
| transferred strand | | |
| Transposon end containing | CTAGTGCATGGGCGCACGAAAAACGCGAAAGCGTTTCACG | 31 |
| internal CATG barcode, non- | ATAAATGCGAAAAC | |
| transferred strand | | |
| Transposon end containing | GTTTTCGCATTTATCGTGAAACGCTTTCGCGTTTTTCGTG | 32 |
| internal TTTC barcode, | CGCCTTTCCA | |
| transferred strand | | |
| Transposon end containing | CTAGTGGAAAGGCGCACGAAAAACGCGAAAGCGTTTCACG | 33 |
| internal TTTC barcode, non- | ATAAATGCGAAAAC | |
| transferred strand | | |
| Transposon end containing | GTTTTCGCATTTATCGTGAAACGCTTTCGCGTTTTTCGTG | 34 |
| internal GGGA barcode, | CGCCGGGACA | |
| transferred strand | | |
| Transposon end containing | CTAGTGTCCCGGCGCACGAAAAACGCGAAAGCGTTTCACG | 35 |
| internal GGGA barcode, non- | ATAAATGCGAAAAC | |
| transferred strand | | |
| Transposon end containing | GTTTTCGCATTTATCGTGAAACGCTTTCGCGTTTTTCGTG | 36 |
| internal CTCTbarcode, | CGCCCTCTCA | |
| transferred strand | | |
| Transposon end containing | CTAGTGAGAGGGCGCACGAAAAACGCGAAAGCGTTTCACG | 37 |
| internal CTCTbarcode, non- | ATAAATGCGAAAAC | |
| transferred strand | | |
| Transposon end containing | GTTTTCGCATTTATCGTGAAACGCTTTCGCGTTTTTCGTG | 38 |
| internal AGAG barcode, | CGCCAGAGCA | |
| transferred strand | | |
| Transposon end containing | CTAGTGCTCTGGCGCACGAAAAACGCGAAAGCGTTTCACG | 39 |
| internal AGAG barcode, non- | ATAAATGCGAAAAC | |
| transferred strand | | |
| Transposon end containing | GTTTTCGCATTTATCGTGAAACGCTTTCGCGTTTTTCGTG | 40 |
| internal ACGC barcode, | CGCCACGCCA | |
| transferred strand | | |
| Transposon end containing | CTAGTGGCGTGGCGCACGAAAAACGCGAAAGCGTTTCACG | 41 |
| internal ACGC barcode, non- | ATAAATGCGAAAAC | |
| transferred strand | | |
| Transposon end containing | GTTTTCGCATTTATCGTGAAACGCTTTCGCGTTTTTCGTG | 42 |
| internal TATA barcode, | CGCCTATACA | |
| transferred strand | | |
| Transposon end containing | CTAGTGTATAGGCGCACGAAAAACGCGAAAGCGTTTCACG | 43 |
| internal TATA barcode, non- | ATAAATGCGAAAAC | |
| transferred strand | | |
| Transposon end containing | GTTTTCGCATTTATCGTGAAACGCTTTCGCGTTTTTCGTG | 44 |
| internal GCGTbarcode, | CGCCGCGTCA | |
| transferred strand | | |
| Transposon end containing | CTAGTGACGCGGCGCACGAAAAACGCGAAAGCGTTTCACG | 45 |
| internal GCGTbarcode, non- | ATAAATGCGAAAAC | |
| transferred strand | | |
| SEQ1 to be introduced within | AGATGTGTATAAGAGACAG | 46 |
| transposon (Tn5 transposon ME | | |
| element) | | |
| SEQ2 to be introduced within | GCTCTTCCGATCT | 47 |
| transposon (3' part of TruSeq | | |
| adaptor) | | |
| Transposon end containing SEQ1 | GTTTTAGATGTGTATAAGAGACAGTTTCGCGTTTTTCGTG | 48 |
| at 6-24 positions, transferred | CGCCGCTTCA | |
| strand | | |
| Transposon end containing SEQ1 | CTAGTGAAGCGGCGCACGAAAAACGCGAAACTGTCTCTTA | 49 |
| at 6-24 positions, non- | TACACATCTAAAAC | |
| transferred strand | | |
| Transposon end containing SEQ2 | GTTTTCGCTCTTCCGATCTAACGCTTTCGCGTTTTTCGTG | 50 |
| at 7-19 positions, transferred | CGCCGCTTCA | |
| strand | | |
| Transposon end containing SEQ2 | CTAGTGAAGCGGCGCACGAAAAACGCGAAAGCGTTAGATC | 51 |
| at 7-19 positions, non- | GGAAGAGCGAAAAC | |
| transferred strand | | |
| Transposon end containing SEQ2 | GTTTTCGCATTTATCGTGAAACGCTTTCGCGTTTTTGCTC | 52 |
| at 37-49 positions, transferred | TTCCGATCTA | |
| strand | | |
| Transposon end containing SEQ2 | CTAGTAGATCGGAAGAGCAAAAACGCGAAAGCGTTTCACG | 53 |
| at 37-49 positions, non- | ATAAATGCGAAAAC | |
| transferred strand | | |
| T3 promoter | AATTAACCCTCACTAAAG | 54 |
| T7 promoter | TAATACGACTCACTATAG | 55 |
| Transposon end containing T3 | GTTTTCGAATTAACCCTCACTAAAGTTCGCGTTTTTCGTG | 56 |
| promoter at 8-25 positions, | CGCCGCTTCA | |
| transferred strand | | |
| Transposon end containing T3 | CTAGTGAAGCGGCGCACGAAAAACGCGAACTTTAGTGAGG | 57 |
| promoter at 8-25 positions, | GTTAATTCGAAAAC | |
| non-transferred strand | | |
| Transposon end containing T3 | GTTTTCGCATTTATCGTGAAACGCTTTCGCAATTAACCCT | 58 |
| promoter at 31-48 positions, | CACTAAAGCA | |
| transferred strand | | |
| Transposon end containing T3 | CTAGTGCTTTAGTGAGGGTTAATTGCGAAAGCGTTTCACG | 59 |
| promoter at 31-48 positions, | ATAAATGCGAAAAC | |
| non-transferred strand | | |
| Transposon end containing T3 | GTTTTCGCATTTATCGTGAAACGCTTTCGCGAATTAACCC | 60 |
| promoter at 32-49 positions, | TCACTAAAGA | |
| transferred strand | | |
| Transposon end containing T3 | CTAGTCTTTAGTGAGGGTTAATTCGCGAAAGCGTTTCACG | 61 |
| promoter at 32-49 positions, | ATAAATGCGAAAAC | |
| non-transferred strand | | |
| Transposon end containing T7 | GTTTTCGCATTTAATACGACTCACTATAGCGTTTTTCGTG | 62 |
| promoter at 12-29 positions, | CGCCGCTTCA | |
| transferred strand | | |
| Transposon end containing T7 | CTAGTGAAGCGGCGCACGAAAAACGCTATAGTGAGTCGTA | 63 |
| promoter at 12-29 positions, | TTAAATGCGAAAAC | |
| non-transferred strand | | |
| Transposon end containing T7 | GTTTTCGCATTTATCGTGAAACGCTTTCGCGTAATACGAC | 64 |
| promoter at 32-49 positions, | TCACTATAGA | |
| transferred strand | | |
| Transposon end containing T7 | CTAGTCTATAGTGAGTCGTATTACGCGAAAGCGTTTCACG | 65 |
| promoter at 32-49 positions, | ATAAATGCGAAAAC | |
| non-transferred strand | | |
| Transposon end containing 26 | NNTTTCGNNNTTNNNNTGNNNCNNTTTCGCGTTTNNNNTG | 66 |
| randomized nucleotides, | NNNCNNNNNA | |
| transferred strand | | |
| Transposon end containing 22 | NNTTTCGNNNTTNNNNTGNNNCNNTTTCGCGTTTTTCGTG | 67 |
| randomized nucleotides, | NNNCNNNNNA | |
| transferred strand | | |
| Transposon end containing 19 | NNTTTCGNNNTTNNNNTGNNNCNNTTTCGCGTTTTTCGTG | 68 |
| randomized nucleotides, | CGCCNNNNNA | |
| transferred strand | | |
| Transposon end containing 14 | NNTTTCGNNNTTNNNNTGNNNCNNTTTCGCGTTTTTCGTG | 69 |
| randomized nucleotides, | CGCCGCTTCA | |
| transferred strand | | |
| Transposon end containing 27 | GTTTTCGNNNTTNNNNTGNNNCNNTTTCGNNNTTNNNNTG | 70 |
| randomized nucleotides, | NNNCNNNNNA | |
| transferred strand | | |
| Transposon end containing 24 | GTTTTCGCATTTNNNNTGNNNCNNTTTCGNNNTTNNNNTG | 71 |
| randomized nucleotides, | NNNCNNNNNA | |
| transferred strand | | |
| Transposon end containing 20 | GTTTTCGCATTTATCGTGNNNCNNTTTCGNNNTTNNNNTG | 72 |
| randomized nucleotides, | NNNCNNNNNA | |
| transferred strand | | |
| Transposon end containing 17 | GTTTTCGCATTTATCGTGAAACNNTTTCGNNNTTNNNNTG | 73 |
| randomized nucleotides, | NNNCNNNNNA | |
| transferred strand | | |
| Transposon end containing 15 | GTTTTCGCATTTATCGTGAAACGCTTTCGNNNTTNNNNTG | 74 |
| randomized nucleotides, | NNNCNNNNNA | |
| transferred strand | | |
| Transposon end containing 8 | GTTTTCGCATTTATCGTGAAACGCTTTCGCGTTTTTCGTG | 75 |
| randomized nucleotides, | NNNCNNNNNA | |
| transferred strand | | |
| Transposon end containing 12 | GTTTTCGNNNTTNNNNTGNNNCNNTTTCGCGTTTTTCGTG | 76 |
| randomized nucleotides, | CGCCGCTTCA | |
| transferred strand | | |
| Transposon end containing 9 | GTTTTCGCATTTNNNNTGNNNCNNTTTCGCGTTTTTCGTG | 77 |
| randomized nucleotides, | CGCCGCTTCA | |
| transferred strand | | |
| Forward primer | CCCACATCCGCTCTAACCGA | 78 |
| Reverse primer | CCCCGCATAAACACCTCTCTT | 79 |
| Primer P5-D501 | AATGATACGGCGACCACCGAGATCTACACTATAGCCTATG | 80 |
| | CGACACTCGTGAAACGCTTTCGCGTTT | |
| Primer P5-D502 | AATGATACGGCGACCACCGAGATCTACACATAGAGGCATG | 81 |
| | CGACACTCGTGAAACGCTTTCGCGTTT | |
| Primer P5-D503 | AATGATACGGCGACCACCGAGATCTACACCCTATCCTATG | 82 |
| | CGACACTCGTGAAACGCTTTCGCGTTT | |
| Primer P7-D701 | CAAGCAGAAGACGGCATACGAGATATTACTCGCGAGGTCG | 83 |
| | AGTGCATGAAACGCTTTCGCGTTT | |
| Primer P7-D702 | CAAGCAGAAGACGGCATACGAGATTCCGGAGACGAGGTCG | 84 |
| | AGTGCATGAAACGCTTTCGCGTTT | |
| Primer P7-D703 | CAAGCAGAAGACGGCATACGAGATCGCTCATTCGAGGTCG | 85 |
| | AGTGCATGAAACGCTTTCGCGTTT | |
| Primer Read 1 | ATGCGACACTCGTTCGTGCGTCAGTTCA | 86 |
| Primer Read 2 | CGAGGTCGAGTGCAGTTCGTGCGTCAGTTCA | 87 |
| Primer Index read | TGAACTGACGCACGAACTGCACTCGACCTCG | 88 |
| RI sequence of wild type Mu | CTTTCGCGTTTTTCGTGCGCCGCTTCA | 89 |
| transposon end sequence | | |
| R2 sequence of wild type Mu | GTTTTCGCATTTATCGTGAAACG | 90 |
| transposon end sequence | | |

### DESCRIPTION OF THE EMBODIMENTS

### I. Definitions

As used herein, “amplification” or “amplifying” refers to invitro methods of making copies of a particular nucleic acid.

As used herein, “a population of nucleic acid fragments” means a collection of DNA fragments, for example, but not limited to, generated from target DNA.

As used herein, “next-generation sequencing” or “NGS” refer to massively parallel sequencing that allows millions of nucleic acids to be sequenced simultaneously. NGS often relies on sequencing-by-synthesis. In some embodiments, NGS comprises a transposition-assisted sequencing template generation methodology in which the transposition reaction results in fragmentation of the target DNA.

As used herein, a “barcode” refers to a short sequence used to uniquely tag or label molecules in a given library. As used herein, a barcode may be a sample barcode or a molecular barcode. A sample barcode comprises a DNA sequence that is attached to the fragments from each sample during library preparation, such that all fragments belonging to a certain sample (for example, an individual cell) or a certain population of nucleic acid fragments will share the same barcode. A molecular barcode comprises a DNA sequence that is attached to all molecules in a certain sample, such that each molecule has a unique barcode within the same sample, i.e. is uniquely tagged. When such molecules are amplified and sequenced, the barcode may be used for correction or elimination of PCR artifacts that could be misread as sequence variants. A molecular barcode may also be known as a unique molecular identifier (UMI). UMI can comprise longer sequence stretches. A barcode may comprise both a sample barcode and a molecular barcode, in such cases a barcode may comprise longer sequence stretches. A barcode may comprise more than one sample barcode, and/or more than one molecular barcode. For example, a pool of barcoded molecules may all have a common sample barcode, while each individual molecule in such pool additionally has one or more unique molecular barcode that may be different among all the molecules.

As used herein, “target DNA” or “target nucleic acid” refers to often unknown nucleic acids that a user wants to sequence, for example by NGS. Target DNA may come from a biological sample or from any sample comprising nucleic acid, including, but not limited to plant, animal or viral material containing DNA or RNA, such as, for example, tissue or fluid isolated from an individual, from preserved tissue, from in vitro cell culture constituents, or from the environment, as well as samples from individual cells. The sequence of the target DNA may be termed a “target sequence.” In contrast, non-target sequences may be needed for various NGS platforms, such as adapters to act as sequencing primers or to associate fragments of target sequence to flow cells, wherein the non-target sequences have known sequences. In some embodiments, known samples of nucleic acids may be used, for example, as part of an assay validation protocol, but in a real-world scenario target DNA is generally unknown.

As used herein, an “adapter” or “adaptor” refers to a non-target nucleic acid component, generally DNA, that provides a means of addressing a nucleic acid fragment to which it is joined. For example, an adapter may comprise a nucleotide sequence that permits identification, recognition, and/or molecular or biochemical manipulation of the DNA to which the adapter is attached.

As used herein, a “transposon” refers to a nucleic acid segment that is recognized by a transposase or an integrase enzyme and that is an essential component of a functional nucleic acid-protein complex (i.e., the transpososome or transposome) capable of mediating transposition. In one embodiment, a minimal nucleic acid-protein complex capable of transposition in a Mu transposition system comprises four MuA transposase protein molecules and a pair of Mu transposon end sequences that are able to interact with MuA.

As used herein, a “transposase” refers to an enzyme that is a component of a functional nucleic acid-protein complex capable of transposition and which is mediating transposition. A transposase may be capable of forming a functional complex with a transposon end-containing composition and catalyzing insertion or transposition of the transposon end-containing composition into the double-stranded nucleic acid with which it is incubated in an in vitro transposition reaction. Exemplary transposases capable of forming transposome complexes with Mu transposon ends and recombinant transposon ends described herein are bacteriophage transposase enzyme from phage Mu, MuA Transposase, such as that available from Thermo Fisher Scientific, HyperMu™ Hyperactive MuA Transposase (EPICENTRE) or other MuA transposases or derivatives thereof.

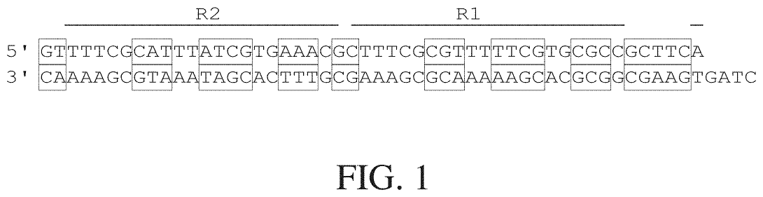

As used herein, “transposon end nucleic acids” or “transposon ends” refers to the nucleotide sequences at the distal ends of a transposon. A transposon end is a double-stranded DNA that exhibits the nucleotide sequences that are necessary to form the functional complex with the transposase or integrase enzyme for use in an in vitro transposition reaction. The transposon end nucleic acids identify the transposon for transposition. The transposase enzyme requires the DNA sequences of the transposon end nucleic acids to form a transpososome complex and perform a transposition reaction, i.e. transposon end nucleic acid is sufficient for transposition event and can be used without the rest of the transposon sequence. A transposon end exhibits two complementary sequences consisting of a “transferred transposon end sequence” or “transferred strand” and a “non-transferred transposon end sequence” or “non-transferred strand.” As shown in FIG. 1, a functional Mu transposon end may comprise a 3′ transposon end's A nucleotide at the transferred strand and a protruding 5′ end at the non-transferred strand. The 3′-end of a transferred strand is joined or transferred to target DNA in an in vitro transposition reaction. In contrast, the non-transferred strand, which exhibits a transposon end sequence that is complementary to the transferred transposon end sequence, is not joined or transferred to the target DNA in an in vitro transposition reaction.

As used herein, an “engineered transposon end” or “recombinant transposon end” nucleic acid refers to a transposon end that is engineered to comprise non-native nucleotide sequence within the transposon end. This transposon end may be referred to as recombinant to indicate that it differs from a wildtype sequence. In some embodiments, the non-native nucleotide sequence is incorporated by making nucleotide substitutions to the recombinant transposon end nucleic acid in comparison to the wild-type sequence. In some embodiments, the recombinant transposon end nucleic acid retains function to associate with a transposase when the non-native nucleotide sequence is incorporated.

As used herein, the “conserved” positions in transposon end nucleic acid sequences were the nucleotide positions that the prior art felt were necessary for activity of transposon end sequences, such as those for binding to transposases (Goldhaber-Gordon JBC 277(10):7703-7712 (2002). As used here, “sensitive” positions are those that had been believed to be the positions, that when substituted with other nucleotides, have a negative effect on transposon binding and activity.

### II. Recombinant Transposon Ends

The MuA transposase recognizes a certain transposon end sequence of 50 base pairs (SEQ ID NO: 1) but is known to tolerate some variation at certain positions. The interaction sites on the transposon DNA are defined by specific DNA sequences (see Goldhaber-Gordon JBC 277(10):7703-7712 (2002)).

This application describes the ability to mutate a significantly larger number of nucleotides than previously described to generate one or more recombinant transposon end nucleic acids, while still retaining function of the transposon end nucleic acids. This increased variability allows for a larger number of individual sequences that can be used as barcodes (enabling barcoding of a larger number of target nucleic acids). Additionally, the recombinant transposon end nucleic acids described in this application allow for additional non-target sequence, such as adapter sequences, to be included within the nucleic acid sequence of the transposon end, instead of needing to incorporate additional non-target sequence information outside of the transposon end, as is done in other methods.

Methodologies to insert barcodes or other sequences into transposon end sequences have been investigated (See, for example US 20150337298; U.S. Pat. No. 9,145,623, and WO 2017/087555). In some cases, previous attempts at using transposon ends comprising barcodes in the generation of DNA sequencing libraries prepared using transposons were limited by the fact that certain nucleic acid positions in the transposon end were considered essential for transposon function, and thus can't be substituted. These presumed essential positions included positions in both the R1 and R2 regions.

In some embodiments, a recombinant transposon end nucleic acid is comprised in a polynucleotide.

In some embodiments, the recombinant transposon end is a Mu transposon end. In some embodiments the wildtype (WT) sequence of the Mu transposon end comprises SEQ ID NO: 1. In some embodiments, the R1 region of the Mu transposon end comprises SEQ ID NO: 89. In some embodiments, the R2 region of the Mu transposon end comprises SEQ ID NO: 90.

In some embodiments, the recombinant transposon end has alterations in the nucleotide sequence of the R1 or R2 region. In some embodiments, the recombinant transposon end nucleic acid has alterations in the nucleotide sequence of both the R1 and R2 regions of the Mu transposon end.

In some embodiments, the recombinant transposon end nucleic acid comprises the nucleotide sequence of SEQ ID NO: 1 having from 15 to 29 nucleotide substitutions at positions selected from 1, 2, 8, 9, 10, 13, 14, 15, 16, 19, 20, 21, 23, 24, 30, 31, 32, 35, 36, 37, 38, 41, 42, 43, 45, 46, 47, 48, 49.

In some embodiments, the recombinant transposon end nucleic acid comprises the nucleotide sequence of SEQ ID NO: 1 having a nucleotide substitution at one or more nucleotide positions selected from among positions 1, 2, 8, 9, 10, 13, 14, 15, 16, 19, 20, 21, 24, 37, 41.

In some embodiments, the recombinant transposon end nucleic acid comprises the nucleotide sequence of SEQ ID NO: 1 having nucleotide substitutions at one or more positions corresponding to positions selected from 1, 2, 8, 9, 10, 13, 14, 15, 16, 19, 20, 21, 23, 24, 37, 41, or 49 positions of SEQ ID NO: 1.

In some embodiments, at least one transposon end nucleic acid has one or more substitution at a sequence corresponding to N positions in SEQ ID NO: 20. In some embodiments, the transposon end nucleic acid further comprises one or more additional nucleotide substitutions.

In some embodiments, a recombinant transposon end nucleic acid comprises nucleotide substitution at position 6, 11, 12, 17, 18, 22, 25, 26 and/or 28, and, optionally, one or more nucleotide substitutions at positions corresponding to N positions in SEQ ID NO: 76. In some embodiments, a recombinant transposon end nucleic acid comprises a variant of the nucleotide sequence of SEQ ID NO: 1 having nucleotide substitutions at positions 6, 12, and 17. In some embodiments, the recombinant transposon end nucleic acid also comprises one or more nucleotide substitutions at positions corresponding to N positions in SEQ ID NO: 76. In some embodiments, a recombinant transposon end nucleic acid comprises nucleotide substitution at positions 11 and 12, and, optionally, one or more nucleotide substitutions at positions corresponding to N positions in SEQ ID NO: 76.

In some embodiments, a recombinant transposon end nucleic acid comprises a variant of the nucleotide sequence of SEQ ID NO: 1 having nucleotide substitutions at positions 12, 18, 22, and 25. In some embodiments, the recombinant transposon end nucleic acid also comprises one or more nucleotide substitutions at positions corresponding to N positions in SEQ ID NO: 76;

In some embodiments, a recombinant transposon end nucleic acid comprises a variant of the nucleotide sequence of SEQ ID NO: 1 having nucleotide substitutions at positions 39, 40, and 44. In some embodiments, the recombinant transposon end nucleic acid also comprises one or more nucleotide substitutions at positions corresponding to N positions in SEQ ID NO: 74. In some embodiments, a recombinant transposon end nucleic acid comprises nucleotide substitution at positions 33, 39, 40, and/or 44, and, optionally, one or more nucleotide substitutions at positions corresponding to N positions in SEQ ID NO: 73.

In some embodiments, a recombinant transposon end nucleic acid comprises nucleotide substitutions at positions 40, and, optionally, one or more nucleotide substitutions at positions corresponding to N positions in SEQ ID NO: 74. In some embodiments, a recombinant transposon end nucleic acid comprises nucleotide substitutions at positions 33 and 40, and, optionally, one or more nucleotide substitutions at positions corresponding to N positions in SEQ ID NO: 74. In some embodiments, a recombinant transposon end nucleic acid comprises a variant of the nucleotide sequence of SEQ ID NO: 1 having nucleotide substitutions at positions 33, 39, and 40. In some embodiments, the recombinant transposon end nucleic acid also comprises one or more nucleotide substitutions at positions corresponding to N positions in SEQ ID NO: 74;

In some embodiments, a recombinant transposon end nucleic acid comprises nucleotide substitution at position 28, and, optionally, one or more nucleotide substitutions at positions corresponding to N positions in SEQ ID NO: 77. In some embodiments, a recombinant transposon end nucleic acid comprises nucleotide substitutions at positions 26, and 28, and, optionally, one or more nucleotide substitutions at positions corresponding to N positions in SEQ ID NO: 77. In some embodiments, a recombinant transposon end nucleic acid comprises a variant of the nucleotide sequence of SEQ ID NO: 1 having nucleotide substitutions at positions 17, 26, and 28. In some embodiments, the recombinant transposon end nucleic acid also comprises one or more nucleotide substitutions at positions corresponding to N positions in SEQ ID NO: 77;

In some embodiments, a recombinant transposon end nucleic acid comprises a variant of the nucleotide sequence of SEQ ID NO: 1 having nucleotide substitutions at positions 33, 34, 39, and 40. In some embodiments, the recombinant transposon end nucleic acid also comprises one or more nucleotide substitutions at positions corresponding to N positions in SEQ ID NO: 16.

In some embodiments, a recombinant transposon end nucleic acid may further comprise one, two, three, four, or five additional nucleotide substitutions compared to the nucleotide sequence of SEQ ID NO: 1.

In some embodiments, a recombinant transposon end nucleic acid comprises nucleotide substitutions that generate one or more additional functions. Non-limiting examples of additional functions include flow cell binding sequences (i.e., platform-specific sequences to bind a library to a sequencing instrument), sequencing primer sites, sample indexes (short sequences specific to a given sample library), and barcodes.

In some embodiments, a recombinant transposon end nucleic acid comprises nucleotide substitutions, wherein the nucleotide substitutions generate a barcode.

In some embodiments, a recombinant transposon end nucleic acid comprises nucleotide substitutions, wherein the nucleotide substitutions generate an additional biological function in the recombinant transposon end nucleic acid. Use of a recombinant transposon end nucleic acid sequence that generates additional biological function may improve or simplify downstream methods compared to use of a wildtype transposon end nucleic acid.

In some embodiments, the additional biological function comprises (i) a primer binding site; (ii) all or part of a restriction endonuclease recognition site; and/or (iii) all or part of a promoter sequence.

A. Barcode

In some embodiments, a recombinant transposon end nucleic acid comprises a barcode.

Barcodes may be used in an NGS protocol to increase error correction and accuracy. Barcodes are short sequences, often with degenerate bases, that incorporate a unique sequence onto different molecules within a given sample library. Barcodes can decrease the rate of false-positive variant calls and thereby increase sensitivity of variant detection. By incorporating individual barcodes onto DNA fragments in a library, variant alleles present in the original sample (i.e., true variants) can be distinguished from errors introduced during library preparation, target enrichment, or sequencing. Thus, barcodes can allow identification and removal of errors by bioinformatics methods before final data analysis, thereby increasing the sensitivity of NGS to identify true variants. In some embodiments, a barcode is a sample barcode to label fragments from each sample during library preparation, such that all fragments belonging to a certain sample (for example, an individual cell) or a certain population of nucleic acid fragments will share the same barcode. In some embodiments, the barcode is a molecular barcode that assigns unique sequences to all molecules from a certain sample. A barcode may comprise both a sample barcode and a molecular barcode, in such cases a barcode may comprise longer sequence stretches. A barcode may comprise more than one sample barcode, and/or more than one molecular barcode. For example, a pool of barcoded molecules may all have a common sample barcode, while each individual molecule in such pool additionally has one or more unique molecular barcode that may be different among all the molecules.

Using the available positions for substitutions disclosed herein, a much broader range of barcodes can be incorporated in a recombinant transposon end nucleic acid. For example, barcodes can be incorporated at different positions of recombinant transposon end nucleic acid sequences than those previously disclosed, or the barcodes may comprise longer sequences than previously disclosed.

B. Primer Binding Site

In some embodiments, a recombinant transposon end nucleic acid comprises a primer binding site (or hybridization site sequences). These primer binding sites may be custom (i.e., designed by the user), PCR primers or commonly-used primers such as known sequencing primers.

In some embodiments, the primer binding site sequence comprises AGATGTGTATAAGAGACAG (SEQ ID NO: 46, comprising a Tn5 transposon mosaic end element) or GCTCTTCCGATCT (SEQ ID NO: 47, comprising 3′ part of TruSeq™ adapter).

C. Restriction Endonuclease Recognition Site

In some embodiments, a recombinant transposon end nucleic acid comprises a restriction endonuclease recognition site. In some embodiments, the restriction endonuclease recognition site exhibits a sequence for the purpose of facilitating cleavage using a restriction endonuclease.

As used herein, a restriction endonuclease is an enzyme that can cleave DNA specifically at a restriction endonuclease binding site. A wide variety of restriction endonucleases are well-known in the art. In some embodiments, the restriction endonuclease is a rate-cutting restriction endonuclease, such as NotI or AscI.

In some embodiments, a restriction endonuclease recognition site is used to generate a compatible double stranded 5′-end in a resulting fragment so that this end can be ligated to another DNA molecule using a template-dependent DNA ligase.

D. DNA-Binding Protein Recognition Sequence

In some embodiments, a recombinant transposon end nucleic acid comprises a DNA-binding protein recognition sequence. In some embodiments, the DNA-binding protein is a DNA-binding protein domain. In some embodiments, the DNA-binding protein is an antibody.

E. Promoter Sequence

In some embodiments, a recombinant transposon end nucleic acid sequence comprises a promoter sequence. As used herein, a “promoter” is a region of DNA that leads to initiation of transcription. In some embodiments, the promoter sequence is a T3 or T7 promoter.

F. Combinations of Substitutions

One skilled in the art will recognize that more than one barcode and/or sequence that generates an additional biological function can be used in a given recombinant transposon end nucleic acid sequence. For example, a given recombinant transposon end nucleic acid sequence can be designed with a barcode and a promoter sequence to allow barcoding and methods using resulting fragments that comprise promoter sequences.

A wide range of recombinant transposon end nucleic acid sequences can be designed to incorporate a combination of substitutions for different purposes. In some embodiments, one set of substitutions is in the R1 region of a recombinant transposon end nucleic acid sequence while another set of substitutions is in the R2 region of a recombinant transposon end nucleic acid sequence.

In some embodiments, substitutions in the R1 region create more than one barcode and/or sequence that generates an additional biological function in the R1 region. In some embodiments, substitutions in the R2 region create more than one barcode and/or sequence that generates an additional biological function in the R2 region.

For example, a recombinant transposon end nucleic acid sequence may comprise a T7 promoter and a sample barcode in the R2 region and a sample barcode in the R1 region. One skilled in the art would understand that a wide range of recombinant transposon end nucleic acid sequences can be designed for a wide range of different uses in NGS based on combinations of substitutions.

In some embodiments, the present substitutions that generate one or more barcode and/or sequence that generates an additional biological function can be combined with other modifications of recombinant transposon end nucleic acids. For example, the present substitutions could be generated in recombinant transposon end nucleic acid sequences also comprising other modifications. In some embodiments, the other modifications may be a nick, gap, apurinic site or apyrimidinic site, such as those described in WO2017087555, which is incorporated by reference herein in its entirety. In some embodiments, recombinant transposon end nucleic acid sequences are pre-nicked and comprise one or more substitutions described herein.

G. Kits

In some embodiments, a kit for use in DNA sequencing is provided. The kit may comprise at least a transposon nucleic acid comprising a recombinant transposon end sequence. In some embodiments, the recombinant transposon end sequence comprised in the kit is a Mu transposon end sequence. In some embodiments, the recombinant transposon end nucleic sequence further comprises a nucleotide sequence that generates an additional biological function in the recombinant transposon end nucleic acid. The kit may also comprise additional components, such as buffers for performing a transposition reaction, control DNA, transposase enzyme, DNA polymerase, DNA cleanup module. The kit can be packaged in a suitable container with instructions for use.

In some embodiments, a buffer comprised in a kit is optimal buffer 1× Fragmentation Reaction Buffer (Thermo Scientific™ MuSeek™ Library Preparation Kit, Illumina™ compatible, Cat. No. K1361).

### III. Composition Comprising a Mixture of Recombinant Transposon End Nucleic Acids

In some embodiments, a composition comprises a mixture of different recombinant transposon ends.

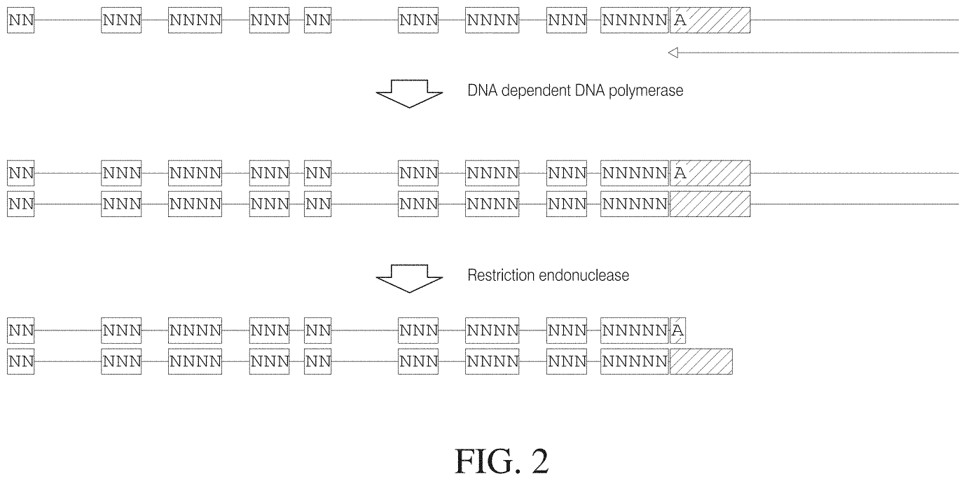

In some embodiments, a composition comprises a mixture of polynucleotides comprising different recombinant transposon ends. In some embodiments, the polynucleotides comprise tags, adapters, primer binding sequences or other sequences, in addition to transposon ends. In some embodiments, the polynucleotides further comprise an extension primer binding site and a restriction endonuclease cutting site at conjunction. In some embodiments, the restriction endonuclease generates a 3′ recessed adenosine (A) and protruding 5′ end with at least 3 or more nucleotides with any base content. In some embodiments, the restriction endonuclease cutting site is HindIII, BcuI, or any other restriction endonuclease known in the art. In some embodiments, the restriction endonuclease cutting site is an isoschizomers such as SpeI, AhlI, or others known in the art. DNA dependent DNA polymerase is used to make a complementary strand. In some embodiments, functional transposon ends are generated using one or more restriction enzyme (See, for example, FIG. 2).

A wide range of substitutions from the wildtype sequence (SEQ ID NO: 1) are shown herein to support function of recombinant transposon end nucleic acids. Up to 29 different positions were shown to have structural function and be permissive for substitutions without severe changes in binding and activity of the transposon end (See FIG. 1).

In some embodiments, a composition comprises a mixture of at least 25 different transposon end nucleic acids. In some embodiments, the mixture comprises at least 25, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000, 11000, 12000, 13000, 14000, 15000, 16000, 17000, 18000, 19000, 20000, 25000, 50000, 75000, 100000, 200000, 300000, 400000, 500000, 600000, 700000, 800000, 900000, 1000000, or more transposon end nucleic acids.

In some embodiments, a composition comprises a mixture of at least 16, 64, 256, 1024, 4096, 16384, 65536, 262144, 1048576, 4194304, 16777216, 67108864, 268435456, 1073741824, 4294967296, 17179869184, 68719476736, or more transposon end nucleic acids.

In some embodiments, each nucleic acid in a mixture is unique.

In some embodiments, a substitution at each N can be independently chosen from A, C, G, and T. In some embodiments, a substitution at an N position can comprise either a pyrimidine or a purine.

Based on the ability to substitute A, C, G, or T at the permissive positions, theoretically up to 4²⁹ different unique recombinant transposon end nucleic acids can be generated that can bind and have activity. This creates an enormous number of unique recombinant transposon end nucleic acids of different sequences.

In some embodiments, a composition comprises a mixture of at least 25 different recombinant transposon end nucleic acids each independently comprising the nucleotide sequence of 5′-NNTTTCGNNNTTNNNNTGNNNCNNTTTCGNNNTTNNNNTGNNNCNNNNNA-3′ (SEQ ID NO: 20); wherein in each nucleic acid each N is independently chosen from A, C, G, and T.

In some embodiments, the mixture of recombinant transposon end nucleic acids comprises the nucleotide sequence of 5′-NNTTTCGNNNTTNNNNTGNNNCNNTTTCGCGTTT NNNNTGNNNCNNNNNA-3′ (SEQ ID NO: 66); wherein in each nucleic acid each N is independently chosen from A, C, G, and T.

In some embodiments, the mixture of recombinant transposon end nucleic acids comprises the nucleotide sequence of 5′-NNTTTCGNNNTTNNNNTGNNNCNNTTTCGCG TTTTTCGTGNNNCNNNNNA-3′ (SEQ ID NO: 67); wherein in each nucleic acid each N is independently chosen from A, C, G, and T.

In some embodiments, the mixture of recombinant transposon end nucleic acids comprises the nucleotide sequence of 5′-NNTTTCGNNNTTNNNNTGNNNCNNTTTCGCG TTTTTCGTGCGCCNNNNNA-3′ (SEQ ID NO: 68); wherein in each nucleic acid each N is independently chosen from A, C, G, and T.

In some embodiments, the mixture of recombinant transposon end nucleic acids comprises the nucleotide sequence of 5′-NNTTTCGNNNTTNNNNTGNNNCNNTTTCGCG TTTTTCGTGCGCCGCTTCA-3′ (SEQ ID NO: 69); wherein in each nucleic acid each N is independently chosen from A, C, G, and T.

In some embodiments, the mixture of recombinant transposon end nucleic acids comprises the nucleotide sequence of 5′-GTTTTCGNNNTTNNNNTGNNNCNNTTTCGCGTTTTTCGTGCGCCGCTTCA-3′ (SEQ ID NO: 76); wherein in each nucleic acid each N is independently chosen from A, C, G, and T.

In some embodiments, the mixture of recombinant transposon end nucleic acids comprises the nucleotide sequence of 5′-GTTTTCGCATTTNNNNTGNNNCNNTTTCGCGTTTTTCGTGCGCCGCTTCA-3′ (SEQ ID NO: 77); wherein in each nucleic acid each N is independently chosen from A, C, G, and T.

In some embodiments, the mixture of recombinant transposon end nucleic acids comprises the nucleotide sequence of 5′-GTTTTCGNNNTTNNNNTGNNNCNNTTTCGNNN TTNNNNTGNNNCNNNNNA-3′ (SEQ ID NO: 70); wherein in each nucleic acid each N is independently chosen from A, C, G, and T.

In some embodiments, the mixture of recombinant transposon end nucleic acids comprises the nucleotide sequence of 5′-GTTTTCGCATTTNNNNTGNNNCNNTTTCGNNN TTNNNNTGNNNCNNNNNA-3′ (SEQ ID NO: 71); wherein in each nucleic acid each N is independently chosen from A, C, G, and T.

In some embodiments, the mixture of recombinant transposon end nucleic acids comprises the nucleotide sequence of 5′-GTTTTCGCATTTATCGTGNNNCNNTTTCGNNN TTNNNNTGNNNCNNNNNA-3′ (SEQ ID NO: 72); wherein in each nucleic acid each N is independently chosen from A, C, G, and T.

In some embodiments, the mixture of recombinant transposon end nucleic acids comprises the nucleotide sequence of 5′-GTTTTCGCATTTATCGTGAAACNNTTTCGNNN TTNNNNTGNNNCNNNNNA-3′ (SEQ ID NO: 73); wherein in each nucleic acid each N is independently chosen from A, C, G, and T.

In some embodiments, the mixture of recombinant transposon end nucleic acids comprises the nucleotide sequence of 5′-GTTTTCGCATTTATCGTGAAACGCTTTCGNNNTT NNNNTGNNNCNNNNNA-3′ (SEQ ID NO: 74); wherein in each nucleic acid each N is independently chosen from A, C, G, and T.

In some embodiments, the mixture of recombinant transposon end nucleic acids comprises the nucleotide sequence of 5′-GTTTTCGCATTTATCGTGAAACGCTTTCGCGTTT NNNNTGNNNCNNNNNA-3′ (SEQ ID NO: 16); wherein in each nucleic acid each N is independently chosen from A, C, G, and T.

In some embodiments, the mixture of recombinant transposon end nucleic acids comprises the nucleotide sequence of 5′-GTTTTCGCATTTATCGTGAAACGCTTTCGCG TTTTTCGTGNNNCNNNNNA-3′ (SEQ ID NO: 75); wherein in each nucleic acid each N is independently chosen from A, C, G, and T.

In some embodiments, the mixture of recombinant transposon end nucleic acids comprises the nucleotide sequence of 5′-GTTTTCGCATTTATCGTGAAACGCTTTCGCG TTTTTCGTGCGCCNNNNNA-3′ (SEQ ID NO: 12); wherein in each nucleic acid each N is independently chosen from A, C, G, and T.

In some embodiments, at least one transposon end nucleic acid has a sequence that has a nucleotide substitution at one or more positions corresponding to positions selected from 1, 2, 8, 9, 10, 13, 14, 15, 16, 19, 20, 21, 23, 24, 37, 41, or 49 positions of SEQ ID NO: 1.

In some embodiments, a composition comprises at least one transposase and a mixture of recombinant transposon end nucleic acids. For example, a composition comprises at least four transposase molecules and a mixture of recombinant transposon end nucleic acids.

### IV. Methods of Use of Recombinant Transposon Ends

Traditional NGS library preparation protocols consisted of three primary steps: fragmentation, adapter ligation, and amplification. Approaches have been investigated to generate fragmentation and tagging (See, for example, EP3272879A1). Advances in methodology, such as Nextera kits (Illumina), have improved this process by combining genome fragmentation and tag addition into a single step, which is termed tagmentation. Tagmentation uses transposons comprising tags to fragment sample DNA and attach the tags to both ends of DNA fragments.

The recombinant transposon ends described in this application can be used in a number of different methods to incorporate biologically relevant functionality during transposition and tagging. In some embodiments, the recombinant transposon end can include one or more adapter sequence. For example, the recombinant transposon end comprising one or more adapter sequence can be used in an ATAC-seq (Buenrostro, 2013) method. In some embodiments, the recombinant transposon end can include a barcode and/or a sequence with an additional biological function. For example, the recombinant transposon end comprising adapter sequence and barcode sequence can be used in a Single-Cell ATAC-seq method.

Similar methods can be performed either with a single recombinant transposon end nucleic acids or a pool thereof.

Methods incorporating adapter sequences within recombinant transposon ends provides for a number of advantages. For example, separate steps of ligating adapters can be avoided in NGS protocols. Decreasing the number of steps in sequencing reactions increases ease of use and reduces reaction time. In addition, reducing steps helps to eliminate errors or variability introduced into the reaction by the end-user, such as pipetting errors.

Further, if adapters are added to target DNA in addition to transposon ends during transposition reactions (as is done in other methods), this increases the size of the final fragments that must be read during sequencing reactions. For example, if an adapter comprising a sequencing primer is placed beyond the transposon end sequence when tagged fragments of target DNA are generated, then the full sequence of the transposon end must be collected each time fragments are sequenced before the target sequence of the fragment can be collected. In other words, when adapters are traditionally used, sequencing primers prime sequencing through the adapters and transposon end before they start to read the target sequence. Thus, very high-quality sequencing reads are wasted on sequencing known sequences before reading unknown sequences from the target nucleic acid. In contrast, recombinant transposon ends that incorporate barcodes, sequencing primers, or other functional biological sequences can reduce this wasted sequencing capacity.

In addition, the availability of a range of N positions available can allow introduction of a longer desired sequence into a recombinant transposon end nucleic acid than previously described. This longer sequence may include, for example, a longer primer sequence or multiple adapters or barcodes.

In some embodiments, a method of fragmenting a sample comprising nucleic acids comprises contacting the sample with one or more recombinant transposon end nucleic acid.

In some embodiments, a method of fragmenting a sample comprising nucleic acids comprises contacting the sample with one or more recombinant transposon end nucleic acid comprising a barcode. In some embodiments, the method further comprises sequencing one or more barcoded nucleic acid fragments. In some embodiments, the sequencing is followed by any of sequence assembly, mutation analysis, allele analysis, copy number analysis, and/or haplotype analysis.

In some embodiments, a method of fragmenting a sample comprising nucleic acids comprises contacting the sample with one or more recombinant transposon end nucleic acid comprising an additional biological function. In some embodiments, the additional biological function comprises (i) a primer binding site; (ii) all or part of a restriction endonuclease recognition site; or (iii) all or part of a promoter sequence.

In some embodiments, a method of fragmenting a sample comprising nucleic acids comprises contacting the sample with one or more recombinant transposon end nucleic acid comprising a primer binding site. In some embodiments, the method further comprises sequencing a fragmented sequence using a primer that binds to the primer binding site. In some embodiments, a sample comprising nucleic acids is contacted with a pool of more than one recombinant transposon end nucleic acid and the fragmented sequences are sequenced with more than one primer.

In some embodiments, a method of fragmenting a sample comprising nucleic acids comprises contacting the sample with one or more recombinant transposon end nucleic acid comprising all or part of a restriction endonuclease binding site. In some embodiments, cleavage at a restriction endonuclease recognition site generates a compatible double stranded 5′-end in the fragment. In some embodiments, the blunt end is ligated to another DNA molecule using a template-dependent DNA ligase.

In some embodiments, the method further comprises cleaving the fragmented sequence with a restriction endonuclease that recognizes the restriction endonuclease binding site. In some embodiments, after reacting the fragmented sequences with a restriction endonuclease, all the fragments comprise similar ends that can be used for ligation reactions. In some embodiments, the ligation reactions add additional nucleic acid sequence to the fragments.

In some embodiments, a method of fragmenting a sample comprising nucleic acids comprises contacting the sample with one or more recombinant transposon end nucleic acid comprising all or part of a promoter sequence. In some embodiments, the promoter sequence is a T3 or T7 promoter. In some embodiments, the method further comprises amplifying the fragmented sequences. In some embodiments, the amplifying is linear amplification. In some embodiments, the linear amplification is in vitro transcription linear amplification, e.g. by using a polymerase capable to perform in vitro transcription using the promoter sequence comprised in the recombinant transposon end nucleic acid sequence. In some embodiments, a polymerase is a T7 RNA polymerase or a derivative thereof. In some embodiments, the method further comprises linear amplification via transposon insertion (LIANTI). In some embodiments, in a method of fragmenting a sample comprising nucleic acids, a recombinant transposon end nucleic acid sequence may comprise a promoter sequence and one or more barcode. One skilled in the art would understand that a wide range of recombinant transposon end nucleic acid sequences can be designed for a wide range of different uses in NGS based on combinations of substitutions.

In some embodiments, the method further comprises reverse transcription and second strand synthesis after linear amplification. In some embodiments, a resulting library is sequenced by NGS after second strand synthesis. In some embodiments, use of a transposon end nucleic acid comprising all or part of a promoter sequence allows generation of a library and sequencing of fragments without requiring a PCR amplification step. In some embodiments, use of a transposon end nucleic acid comprising all or part of a promoter sequence allows generation of a library and sequencing of fragments without requiring exponential amplification.

### V. Methods of Use of Mixtures of Transposon Ends Comprising Random Sequences

In some embodiments, a method of fragmenting a sample comprising nucleic acids comprises contacting the sample with a composition comprising a mixture of at least 25 different recombinant transposon end nucleic acids. In some embodiments, the sample is obtained from one cell.

In some embodiments, a method of generating a population of uniquely barcoded nucleic acid fragments from a sample comprising nucleic acids comprises contacting the sample with a composition comprising a mixture of recombinant transposon end nucleic acids, wherein the composition comprises at least 25, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000, 11000, 12000, 13000, 14000, 15000, 16000, 17000, 18000, 19000, 20000, 25000, 50000, 75000, 100000, 200000, 300000, 400000, 500000, 600000, 700000, 800000, 900000, 1000000, or more transposon end nucleic acids with different sequences.

In some embodiments, a method of generating a population of uniquely barcoded nucleic acid fragments from a sample comprising nucleic acids comprises contacting the sample with a composition comprising a mixture of recombinant transposon end nucleic acids, wherein the composition comprises at least 16, 64, 256, 1024, 4096, 16384, 65536, 262144, 1048576, 4194304, 16777216, 67108864, 268435456, 1073741824, 4294967296, 17179869184, 68719476736, or more transposon end nucleic acids with different sequences.

The utility of barcodes in recombinant transposon end nucleic acid sequences have been described above for sequences having specific substitutions and combinations of substitutions. The use of mixtures of recombinant transposon end nucleic acids can allow generation of an even greater number of unique barcodes.

In some embodiments, a method of generating a population of barcoded nucleic acid fragments from a sample comprising nucleic acids comprises contacting the sample with a composition comprising a mixture of recombinant transposon end nucleic acids. In some embodiments, the recombinant transposon end nucleic acids barcode the nucleic acid fragments from the sample.

In some embodiments, the sequences of the barcodes are used to identify unique fragments generated during fragmentation of the sample. In some embodiments, the method further comprises sequencing the population of barcoded nucleic acid fragments. In some embodiments, the sequencing is followed by any of sequence assembly, mutation analysis, allele analysis, copy number analysis, and/or haplotype analysis.

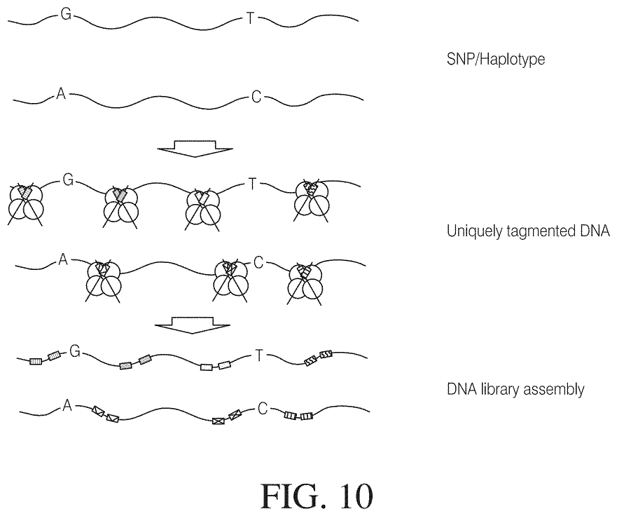

In some embodiments, the sequences of the barcodes are used for realignment of sequences in haplotype analysis. FIG. 10 presents a non-limiting example of how unique sequences, such as barcodes, can be inserted via recombinant transposon ends to help assemble a primary sequence.

In some embodiments, a method of generating a population of uniquely barcoded nucleic acid fragments from a sample comprising nucleic acids further comprises sequencing the population of barcoded nucleic acid fragments. In case when longer barcodes (UMIs) are incorporated during tagmentation, such method can be used to detect rare mutations by reducing sequencing background. For example, DNA polymerase fidelity can be measured using this method. In some embodiments, transposome complexes comprising transposon end nucleic acids with UMIs, that are, for example, 8-16 nt long, are used in such method. For example, recombinant transposon ends of current disclosure that are in transposome complex with MuA transposase may be used.

PCR is performed to amplify a target DNA sequence, with a polymerase of interest. PCR product may be purified from the reaction mixture. Purified PCR product is premixed with transposome complex in a suitable reaction buffer. After fragmentation with transposase, fragmented DNA containing UMIs may be subjected to size selection cleanup. Fragmented DNA may be subjected to PCR amplification to introduce adapters and library barcodes required by the sequencing system to be used. Amplified library may be purified from the reaction mixture. After preparation the libraries are sequenced.

Generated sequencing data can be analyzed by grouping reads to barcode (UMI) families and then calling polymerase errors. Polymerase errors are called only if they are present in all reads in the UMI family, otherwise they are discarded as sequencing error.

DNA that does not undergo the amplification with a polymerase of interest, can be used as a control to evaluate background errors potentially introduced during PCR amplification and sequencing steps.

In some embodiments, where the method is used to detect rare mutations present in the target DNA, the DNA is premixed with transposome complex in a suitable reaction buffer. Transposome complexes comprising transposon end nucleic acids with UMIs, that are, for example, 8-16 nt long, may be used in such method. For example, recombinant transposon ends of current disclosure that are in transposome complex with MuA transposase may be used. After fragmentation with transposase, fragmented DNA containing UMIs may be subjected to size selection cleanup. Fragmented DNA is subjected to PCR amplification to introduce adapters and library barcodes required by the sequencing system to be used. Amplified library may be purified from the reaction mixture. After preparation the libraries are sequenced.

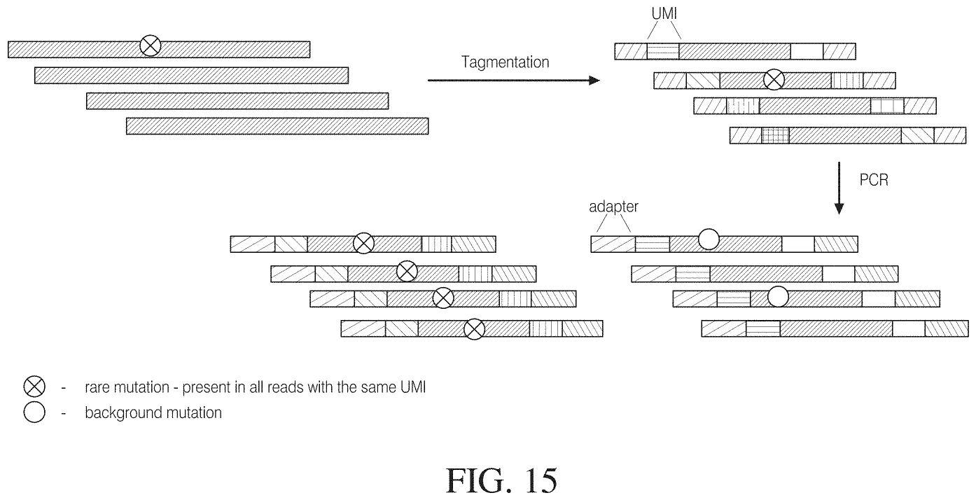

Generated sequencing data can be analyzed by grouping reads to barcode (UMI) families and then calling errors. Errors are called only if they are present in all reads in the UMI family, otherwise they are discarded as sequencing error. The principle of the method for detection of rare mutations is provided in FIG. 15.

DNA known not to contain mutations of interest can be used as a control to evaluate background errors potentially introduced during PCR amplification and sequencing steps.

The described methods for detecting rare mutations and/or for measuring DNA polymerase fidelity can be used with a transposase enzyme, including a DDE transposase enzyme such as a prokaryotic transposase enzyme from ISs, Tn3, Tn5, EZ-Tn5™ hyperactive Tn5 Transposase (EPICENTRE), Tn7, and Tn10, bacteriophage transposase enzyme from phage Mu, MuA Transposase, such as that available from Thermo Fisher Scientific, HyperMu™ Hyperactive MuA Transposase (EPICENTRE) in combination with corresponding transposon ends carrying randomized (UMI) sequence inside or outside transposon sequence.

### EXAMPLES

### Example 1. Evaluation of Transposon End Sequences Comprising Random Sequences (Randomers)

FIG. 1 shows non-conserved region distribution within a Mu transposon end, with the boxed regions indicating nucleotides that were randomized. Random sequences within a transposon end were introduced by employing a template containing transposon end sequence with optimized deoxynucleotide ratio (to yield optimal G:T:A:C 25:25:25:25 randomization level, or any other) and an extension primer binding site, with a restriction endonuclease cutting site at conjunction (FIG. 2). Restriction endonuclease can be any, that generates 3′ recessed adenosine (A) and protruding 5′ end with at least 3 or more nucleotides with any base content. This includes examples such as HindIII, BcuI or any other or their isoschizomers such as SpeI, AhlI, etc. DNA dependent DNA polymerase is used to make a complementary strand. Functional transposon ends are generated using mentioned restriction enzymes.

To generate randomized pre-transposon end first, each of the transposon end templates Mu-NO-temp (control, without randomers; SEQ ID NO: 3), Mu-N5-temp (5 randomers; SEQ ID NO: 4), Mu-N12-temp (12 randomers; SEQ ID NO: 5) and Mu-N29-temp (29 randomers; SEQ ID NO: 6) were annealed in pairs with an extension primer Mu-N-ext (SEQ ID NO 7):
Mu-NO-temp and Mu-N-ext or
Mu-N5-temp and Mu-N-ext or
Mu-N12-temp and Mu-N-ext or
Mu-N29-temp and Mu-N-ext.

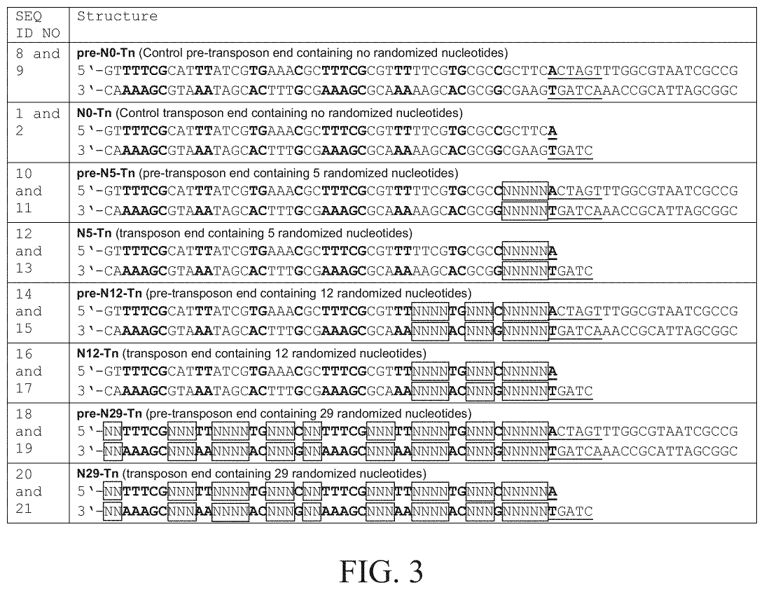

Structures and sequences of pre-transposon ends and corresponding transposon ends are provided in FIG. 3.

Annealing was performed in 50 μL volume at equimolar oligo final concentration of 80 μM in annealing buffer (10 mM Tris-HCl (pH 8.0), 1 mM EDTA, 50 mM NaCl) by heating at 95° C. for 5 minutes, then a minute for each temperature lower by 5° C. until it reaches 5° C. Klenow exo-polymerase (Thermo Scientific, Cat. No. EP0421), buffer, and dNTPs were added to a final 400 μL reaction composition of 50 mM Tris-HCl (pH 8.0), 5 mM MgCl2, 1 mM DTT, 0.25 mM, 100 U Klenow exo-polymerase. The reaction was carried out in 37° C. for 60 minutes.

Each reaction product was purified using Collibri™ Library Cleanup Kit (Invitrogen, Cat. No. A38584096). Each reaction mix was purified in four 100 μL aliquots in 1.5 mL tubes. A volume of 200 μL of thoroughly mixed magnetic cleanup beads together with 200 μL 96% ethanol were added to each tube and mixed well by vortexing. Samples were incubated for fifteen minutes at room temperature. After a short spin, the tubes were placed in a magnetic rack until the solutions were cleared. The supernatant was aspirated carefully without disturbing the beads and discarded. The tubes were kept in the magnetic rack, and 200 μL of freshly prepared 85% ethanol was added. After 30 seconds of incubation, the supernatant was removed. The tubes were given a short spin to collect excess ethanol, which was then removed by a pipette. The beads were then air-dried by opening the tube caps for two minutes, allowing remaining ethanol to evaporate. The tubes were removed from the magnetic rack, and the beads were resuspended in 50 μL of elution buffer (10 mM Tris-HCl (pH 8.3)) by vortexing. The tubes were then placed back in the magnetic rack. After the solution became clear, all supernatants containing double stranded pre-transposon end were carefully transferred into new sterile tubes, where eluates of initially aliquoted samples were combined into the same tube. This yields 200 μL of each pre-transposon end.

Functional transposon ends were then generated in following reaction using 450 U Anza 3 BcuI restriction endonuclease in 300 μL of Anza buffer (Invitrogen, Cat. No. IVGN0036), and incubating at 37° C. for 120 minutes.

Each reaction product was purified using Collibri Library Cleanup Kit (Invitrogen, Cat. No. A38584096). Each reaction mix was purified in three 100 μL aliquots in 1.5 mL tubes. A 200 μL volume of thoroughly mixed magnetic cleanup beads together with 200 μL 96% ethanol were added to each tube and mixed well by vortexing. Samples were incubated for fifteen minutes at room temperature. After a short spin, the tubes were placed in a magnetic rack until the solutions were cleared. The supernatant was aspirated carefully without disturbing the beads and discarded. The tubes were kept in the magnetic rack, and 200 μL of freshly prepared 85% ethanol was added. After 30 seconds of incubation, the supernatant was removed. The wash procedure was repeated. The tubes were given a short spin to collect excess ethanol, which was then removed. The beads were then air-dried by opening the tube caps for two minutes, allowing remaining ethanol to evaporate. The tubes were removed from the magnetic rack, and the beads were suspended in 17 μL of elution buffer (10 mM Tris-HCl (pH 8.3)) by vortexing. The tubes were then placed back in the magnetic rack. After the solution became clear, all supernatants containing transposon ends (17 μL) were carefully transferred into new sterile tubes, where eluates of initially aliquoted samples were combined into the same tube. This yields 50 μL of each transposon end.

Absorption of purified samples was determined using Nano Drop spectrophotometer (Thermo Scientific). Molar concentration was calculated for each transposon end, and a final dilution of 60 μM was prepared.

MuA transposomes were formed in 30 mM Tris-HCl, pH 6.0, 10% (v/v) glycerol, 0.005% (w/v) Triton X-100, 30 mM NaCl, 0.02 mM EDTA, and 10% DMSO. The complex assembly reaction contained equimolar ratio of transposon end (11.2 μM) and MuA transposase (1.65 mg/mL). Components were well mixed and incubated for one hour at 30° C. After incubation, the complex assembly mix was diluted with dilution buffer (88.0% glycerol, 314.5 mM NaCl, and 2.83 mM EDTA) to the final MuA concentration of 0.919 mg/mL. Complexed MuA transposome was stored at −70° C. for at least 16 hours before use.

Complex assembly efficiency was evaluated using an electrophoretic mobility shift assay (EMSA) on a 2% agarose gel containing 0.5 μg/mL ethidium bromide and 87 μg/mL BSA and heparin. Activity was evaluated by fragmenting 100 ng Escherichia virus lambda genomic DNA with 1.5 μL MuA complex in 30 μL of 1× Fragmentation Reaction Buffer (Thermo Scientific™ MuSeek™ Library Preparation Kit, Illumina™ compatible, Cat. No. K1361). Fragmentation was carried out for 5 minutes at 30° C., then stopped by adding 4.4% SDS solution.

Samples were then purified using GeneJET NGS Cleanup Kit (Thermo Scientific, Cat. No. K0851) and collected in 25 μL Elution Buffer. Undiluted samples were analyzed on Agilent Bioanalyzer 2100 (Agilent, Cat. No. G2939BA) using Agilent High Sensitivity DNA Kit (Agilent, Cat. No. 5067-4626).

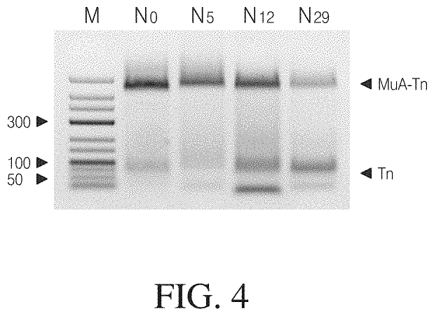

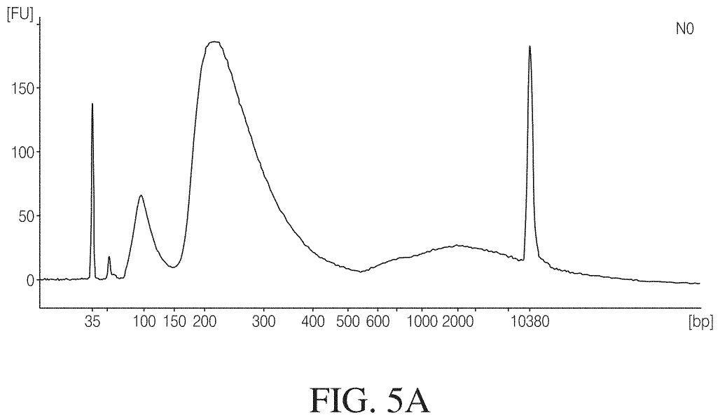

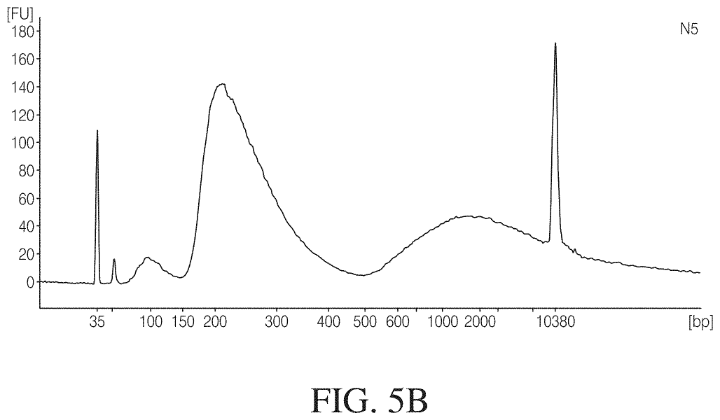

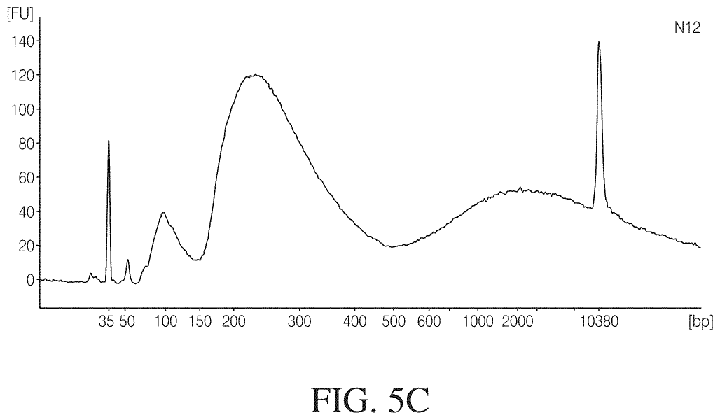

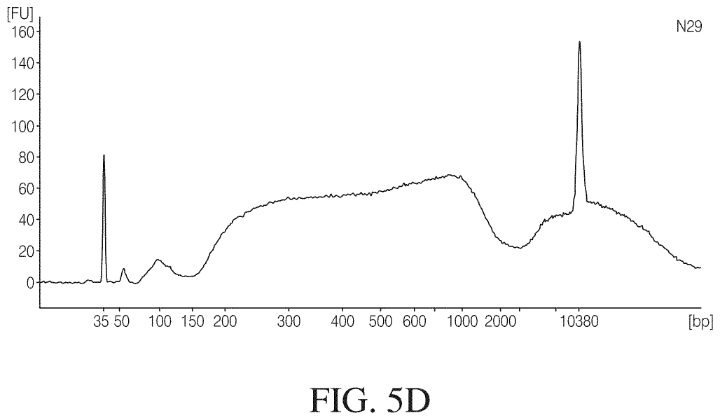

Transposon ends carrying randomized nucleotides at various levels (0 to 29 nucleotides) were able to bind to MuA and form stable transposomes (FIG. 4, highly shifted DNA bands). FIGS. 5A-5D show the activity of transposome complexes carrying transposon ends with various level of randomization. These results indicate that a desired fragmentation profile can be well-controlled by varying a concentration of a complexed MuA transposase.

Therefore, up to 29 nucleotides can be altered within the non-conserved regions of Mu transposon end. The nucleotide content can be altered without dramatic changes in binding and activity. The result shows that MuA transposase tolerates a random nucleotide at certain position, and can equally tolerate any of each individual nucleotides—G, T, C or A.

### Example 2. Use of Transposon End Sequences Comprising Barcodes

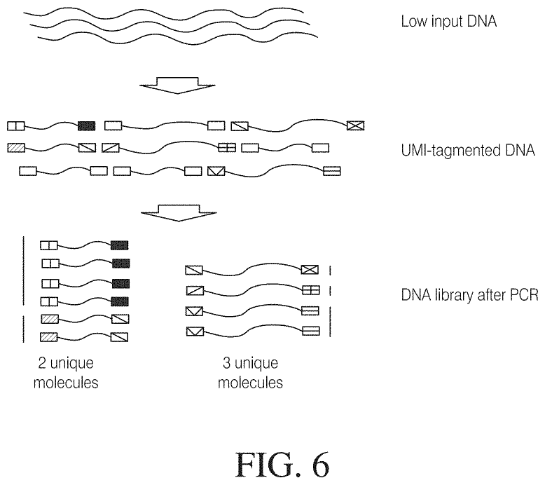

MuA transposase binds randomized sequences carrying transposon ends in a random manner, therefore each transposome complex contains two transposon ends with unique sequences (heterotransposome) or the same sequence (homotransposome), which can be interpreted as a barcode. By employing this kind of randomized transposomes, a nucleic acid can be tagmented, and unique sequences are introduced at both ends of each fragment of tagmented DNA. After a number of PCR cycles to amplify DNA targets and sequencing, reads that align to the same coordinates of a reference can be grouped into those that were unique (carry unique barcodes) and eliminate the effect of PCR duplicates (i.e., reads that contain the same pair of barcodes). A schematic overview of barcode (UMI or molecular barcode) utility is shown in FIG. 6.

PCR can lead to preferential amplification of certain fragments. As shown in FIG. 6, a pool of 6 fragments that comprises 2 unique molecules (i.e., fragments) can be identified based on the presence of the unique UMIs at the opposite ends of the fragments (shown by the differently patterned boxes). A different pool of 4 fragments that comprises 3 unique molecules (i.e., fragments) can be identified based on the presence of the unique UMIs at the opposite ends of the fragments (shown by the differently patterned boxes). In this way, molecular barcodes (or UMIs) can be used to identify sequenced fragments that are copies of the same fragment generated during tagmentation.

### Example 3 Evaluation of Transposon Ends Comprising Unique Barcodes

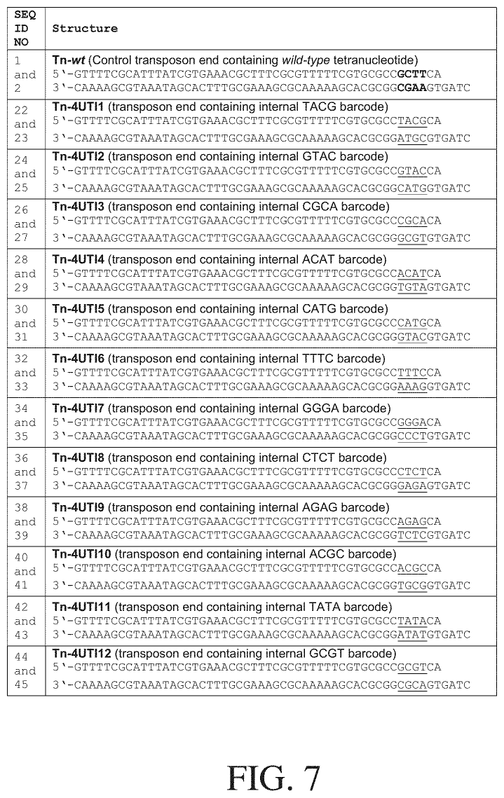

Four nucleotides of a Mu transposon end at positions 45-48 were substituted with unique tetramers. These unique tetramers can be used as barcodes. The result of MuA tolerating a random nucleotide at certain position means that a transposon end sequence can equally tolerate any of each individual nucleotides—G, T, C or A. Transposon ends with barcodes were prepared and then used to make unique sequence carrying MuA transposome complexes. FIG. 7 presents transposon end nucleic acid sequences that were tested. Tetranucleotides in the barcodes were chosen by a rule, wherein sequences have to differ by at least 2 nucleotides across all tetramers. Transposon ends at a final concentration of 60 μM were prepared by annealing equimolar quantities of primers in the pairs as indicated in FIG. 7

Annealing was performed in annealing buffer (10 mM Tris-HCl (pH 8.0), 1 mM EDTA, 50 mM NaCl) by heating at 95° C. for 5 minutes, then a minute for each temperature lower by 5° C. until the temperature reached 5° C.

MuA transposomes were formed in 1× Complex Assembly Buffer with DMSO. The complex assembly reaction contained equimolar ratio of transposon end (9.3 μM) and MuA transposase (1.65 mg/mL). Components were well-mixed and placed for incubated for one hour at 30° C. After incubation, the complex assembly mix was diluted with dilution buffer (88.0% glycerol, 314.5 mM NaCl, and 2.83 mM EDTA) to the final MuA concentration of 0.919 mg/mL. Complexed MuA transposome was stored at −70° C. for at least 16 hours before use.

Complex assembly efficiency was evaluated using an electrophoretic mobility shift assay (EMSA) on a 2% agarose gel containing 0.5 μg/mL Ethidium bromide and 87 μg/mL BSA and heparin. Activity was evaluated by fragmenting 100 ng Escherichia virus lambda genomic DNA with 1.5 μL MuA complex in 1× Fragmentation Reaction Buffer (Thermo Scientific™ MuSeek™ Library Preparation Kit, Illumina™ compatible, Cat. No. K1361). Fragmentation was carried out for 5 minutes at 30° C., then stopped by adding 4.4% SDS solution.

Samples were then purified using GeneJET NGS Cleanup Kit (Thermo Scientific, Cat. No. K0851) and collected in 25 μL Elution Buffer. Undiluted samples were analyzed on Agilent Bioanalyzer 2100 (Agilent, Cat. No. G2939BA) using Agilent High Sensitivity DNA Kit (Agilent, Cat. No. 5067-4626).

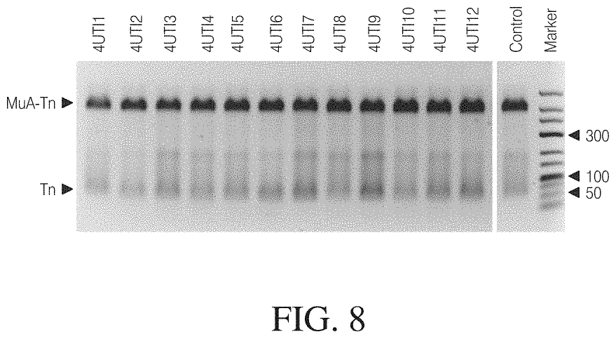

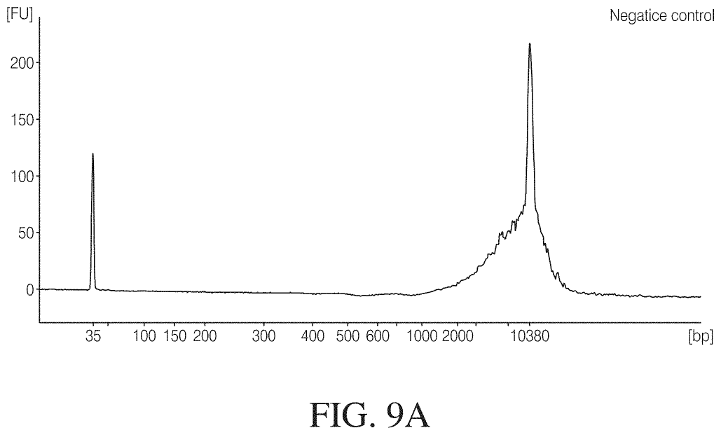

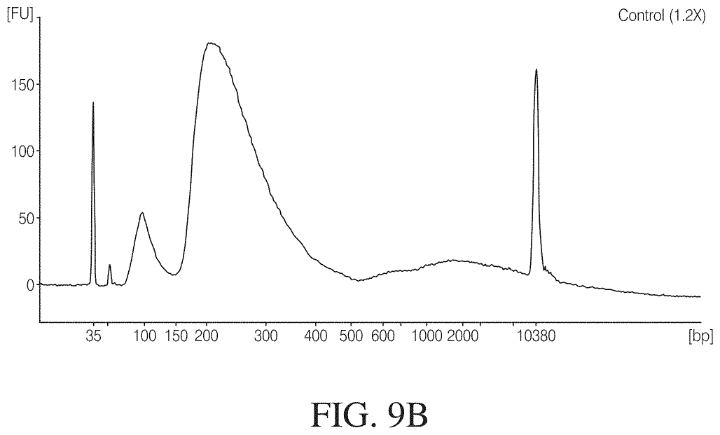

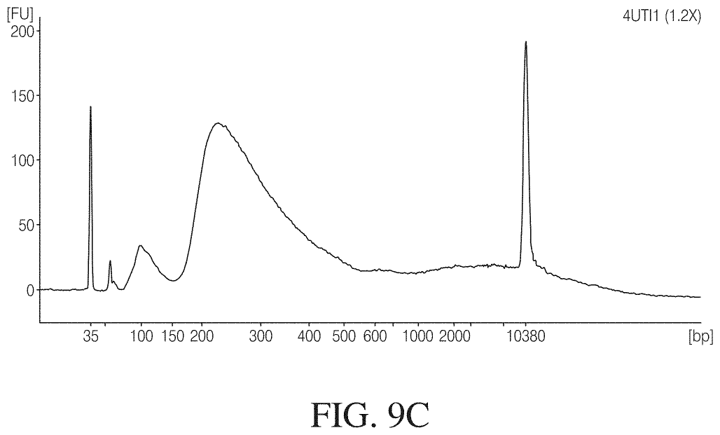

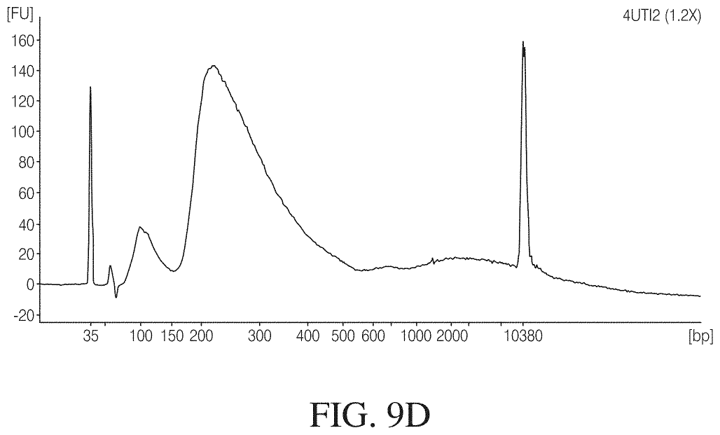

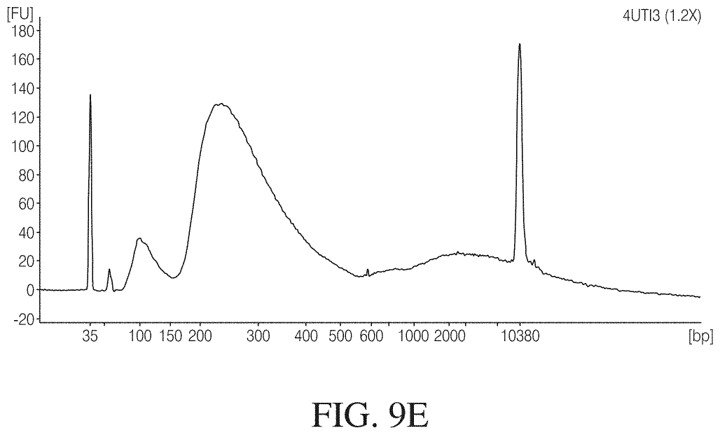

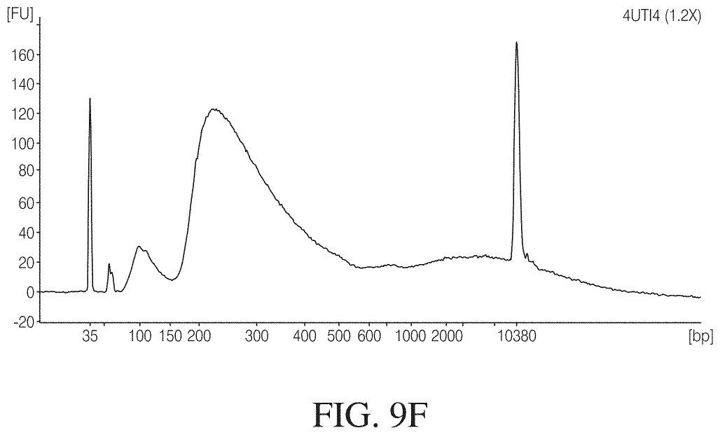

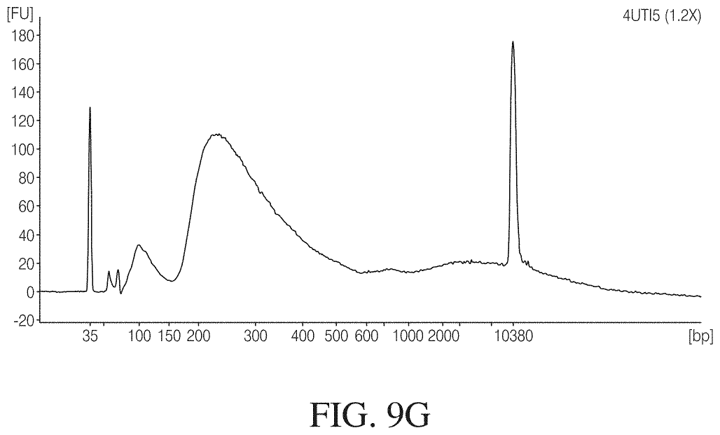

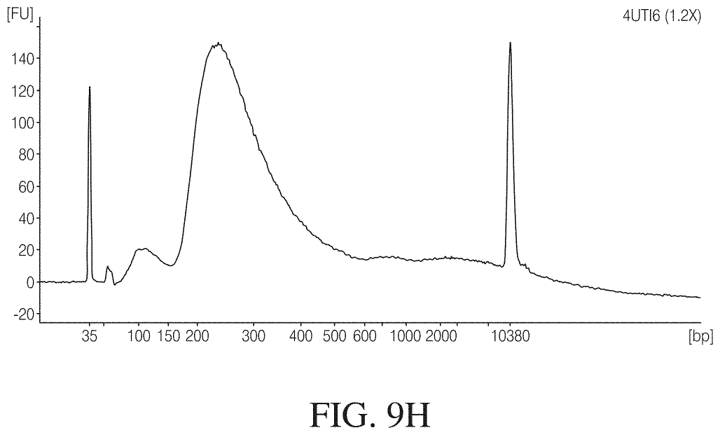

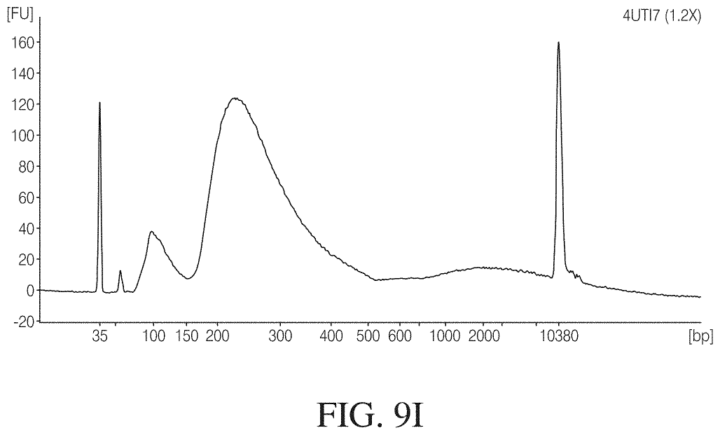

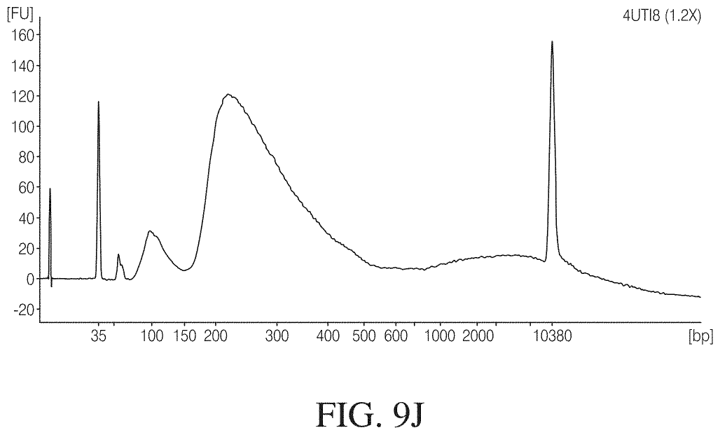

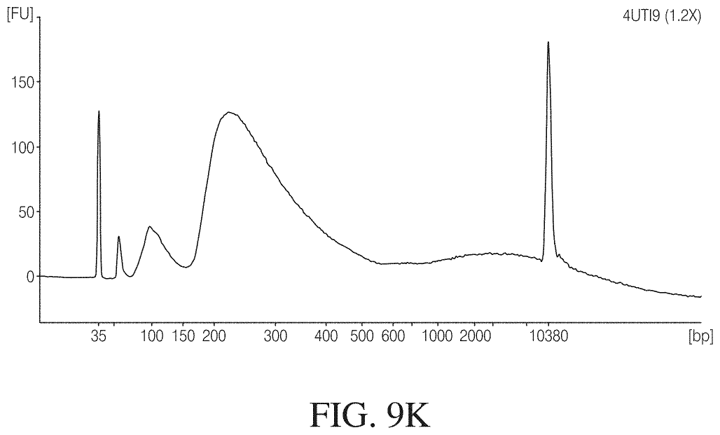

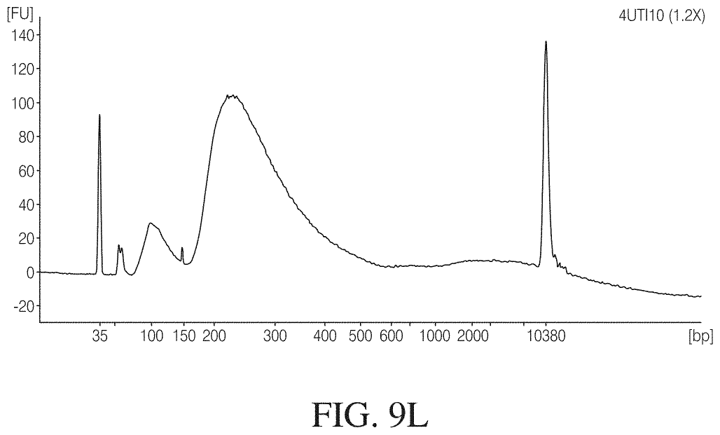

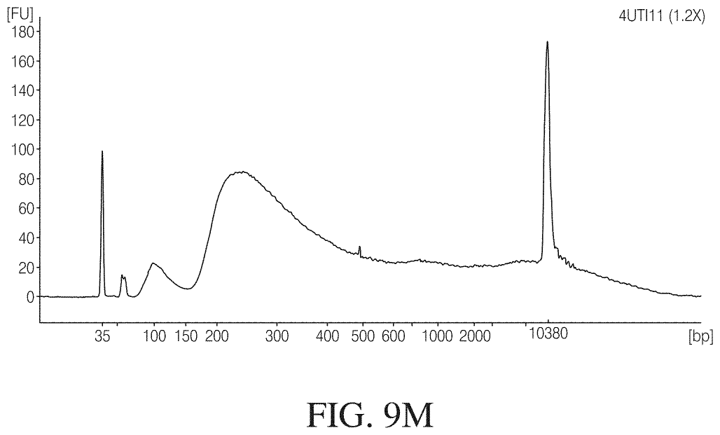

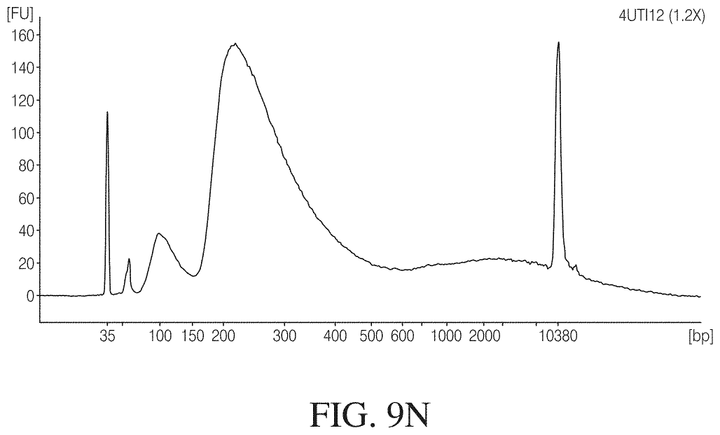

Transposon ends carrying unique tetramer sequences, regardless of the nucleotide content of the tetramer sequence, can bind to MuA (FIG. 8) and form stable transposomes (FIGS. 9A-9N, highly shifted DNA bands). Therefore, introduction of barcodes did not eliminate function of the transposon ends.

### Example 4. UTI Utility in Haplotype Assembly

Transposon end sequences can also be used to generate a primary sequence.

MuA transposase complexes containing unique sequences can be prepared in separate vials, with each transposome complex containing two transposon ends with the same unique sequence. Unique sequences can comprise up to 29 bp; alternatively, more bps can be included with affected activity. These unique sequences can be referred to as a UTI—unique transposon end identifier. A number of transposome complexes (2, 12, 48, 96, 384 or more) may be prepared in such manner and pooled together to yield a pool of transposomes that carry the same UTI within a transposome complex (homotransposome) but differs from any other MuA complex.

By employing this kind of randomized transposases, a nucleic acid can be tagmented and unique tagging sequences are introduced at both ends of each fragment of tagmented DNA, yet preserving a contiguity by having the same UTI sequence at the site of transposition. This allows use of information on the unique sequence of a nucleic acid cleavage site to join ends of two fragments and assemble a primary sequence. A schematic overview of UTI utility is shown in FIG. 10.

### Example 5. Evaluation of Transposon Ends Containing Custom Primer Hybridization Sites

These results indicate that up to 29 nucleotides can be altered within the non-conserved regions of Mu transposon end. This concept allows introduction of custom, non-Mu-native nucleotide sequences to a Mu transposon end which can be used as an oligonucleotide hybridization sites for further applications, such as PCR.

Several transposon ends and complementary sequences were prepared that comprise either hybridization site sequence 1: AGATGTGTATAAGAGACAG (SEQ ID NO: 46) or hybridization site sequence 2: GCTCTTCCGATCT (SEQ ID NO: 47).

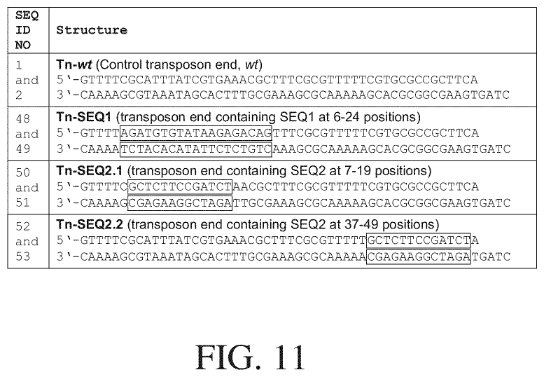

FIG. 11 presents oligonucleotides used to generate custom primer binding sites introduced to a Mu transposon end.

Table 3 presents structural changes of Mu transposon end when custom sequences are introduced. Italics show site of introduced primer binding site. Letters in bold stand for conserved nucleotides. Underlines mean a change is introduced, compared to a wild type transposon end sequence. Boxed letters symbolize changes done in conserved sites and, thus, are called sensitive.

TABLE 3
Structural changes of Mu transposon end when custom sequences are introduced.

| SEQ | Trans- | | |
|---|---|---|---|
| ID | poson | | |
| NO | end | Structure | Substitutions |
| 1 and 2 | Tn-wt | 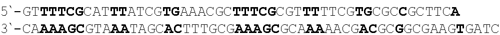 | — |
| 48 and 49 | Tn-SEQ1 | 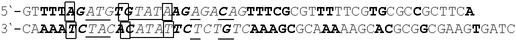 | 13 substitutions (3 sensitive) |
| 50 and 51 | Tn- SEQ2.1 | 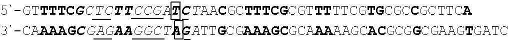 | 8 substitutions (1 sensitive) |
| 52 and 53 | Tn- SEQ2.2 | 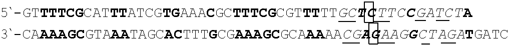 | 8 substitutions (1 sensitive) |

Transposon ends at a final concentration of 60 μM were prepared by annealing equimolar quantities of primers in pairs as provided in Table 3.

Annealing was performed in annealing buffer (10 mM Tris-HCl (pH 8.0), 1 mM EDTA, 50 mM NaCl) by heating at 95° C. for 5 minutes, then a minute for each temperature lower by 5° C. until the temperature reached 5° C.

MuA transposomes were formed in 1× Complex Assembly Buffer with DMSO. Complex assembly reaction contained equimolar ratio of transposon end (9.3 μM) and MuA transposase (1.65 mg/mL). Components were well-mixed and incubated for one hour at 30° C. After incubation, the complex assembly mix was diluted with dilution buffer (88.0% glycerol, 314.5 mM NaCl, and 2.83 mM EDTA) to the final MuA concentration of 0.919 mg/mL. Complexed MuA transposome was stored at −70° C. for at least 16 hours before use.

Complex assembly efficiency was evaluated using an electrophoretic mobility shift assay (EMSA) on a 2% agarose gel containing 0.5 μg/mL Ethidium bromide and 87 μg/mL BSA and heparin. Activity was evaluated by fragmenting 100 ng Escherichia virus lambda genomic DNA with 1.5 μL MuA complex in 1× Fragmentation Reaction Buffer (Thermo Scientific™ MuSeek™ Library Preparation Kit, Illumina™ compatible, Cat. No. K1361). Fragmentation was carried out for 5 minutes at 30° C., then stopped by adding 4.4% SDS solution.

Samples were then purified using GeneJET NGS Cleanup Kit (Thermo Scientific, Cat. No. K0851) and collected in 25 μL Elution Buffer. Undiluted samples were analyzed on Agilent Bioanalyzer 2100 (Agilent, Cat. No. G2939BA) using Agilent High Sensitivity DNA Kit (Agilent, Cat. No. 5067-4626).

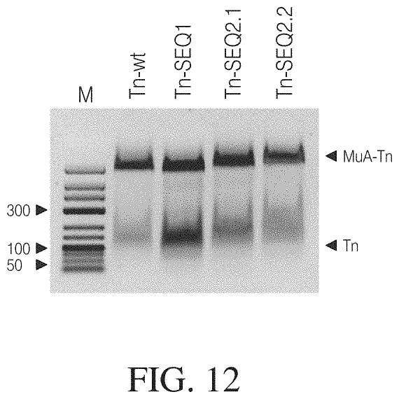

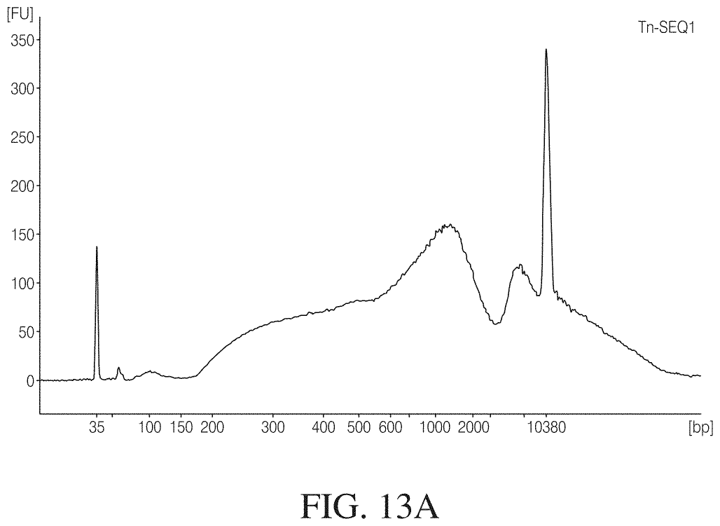

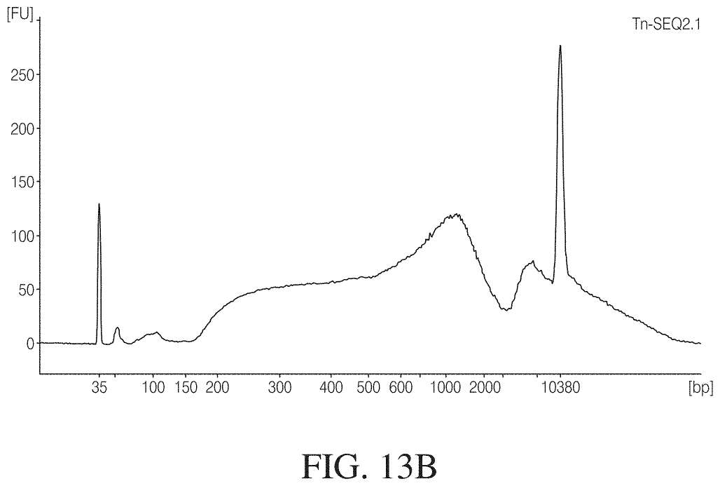

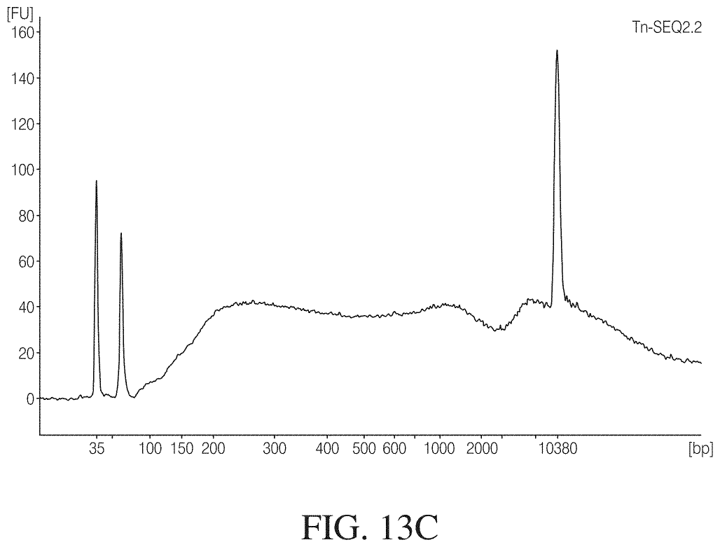

Transposon ends carrying artificial sequences, regardless the substituted nucleotide sequence, are capable to bind to MuA and form stable transposomes (FIG. 12, highly shifted DNA bands). FIGS. 13A-13C shows the activity of transposome complexes carrying transposon ends with various artificial sequences introduced within a Mu transposon end sequence. Even with substitutions at conserved regions, transposases retain high activity level.

### Example 6. Transposon Ends Containing Functional Biological Sequences

The ability to change transposon end sequence (even with some tolerance within conserved region) would allow introduction of a biological sequences that may be used in downstream procedures, such as promoters T3, T7, or any other.

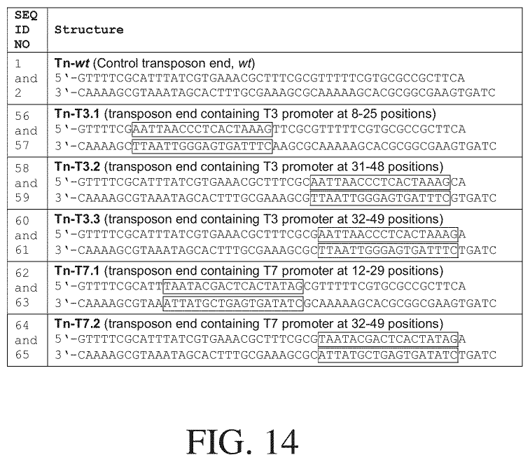

Several transposon end sequences are proposed comprising T3 or T7 promoters and their complementary sequences (FIG. 14, showing T3 or T7 promoter sequences and their complementary sequences in boxes). The T3 promoter sequence is AATTAACCCTCACTAAAG (SEQ ID NO: 54), and T7 promoter sequence is TAATACGACTCACTATAG (SEQ ID NO: 55).

Table 5 presents exemplary transposon end nucleic acid sequences incorporating promoter sequences. Italics show site of introduced primer binding site. Letters in bold stand for conserved nucleotides. Underlines mean changes introduced, compared to a native transposon end sequence. Boxed letters symbolize changes done in conserved sites and, thus, are called sensitive.

TABLE 5
Structural changes of Mu transposon end when functional biological sequences are introduced.

| Trans- | SEQ | | |
|---|---|---|---|
| poson | ID | | |
| end | NO | Structure | Substitutions |
| Tn-wt | 1 and 2 | 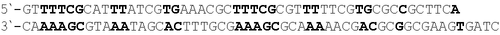 | — |
| Tn-T3.1 | 56 and 57 | 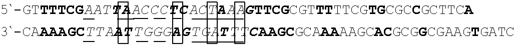 | 11 substitutions (4 sensitive) |
| Tn-T3.2 | 58 and 59 | 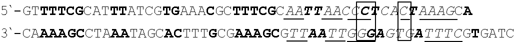 | 13 substitutions (3 sensitive) |
| Tn-T3.3 | 60 and 61 | 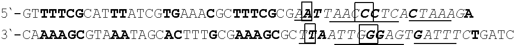 | 15 substitutions (3 sensitive) |
| Tn-T7.1 | 62 and 63 | 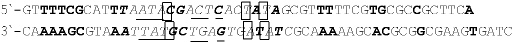 | 9 substitutions (3 sensitive) |
| Tn-T7.2 | 64 and 65 | 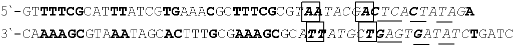 | 12 substitutions (4 sensitive) |
| Tn-T7.3 | 103 and 104 | 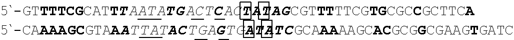 | 9 substitutions (2 sensitive) |
| Tn-T7.4 | 105 and 106 | 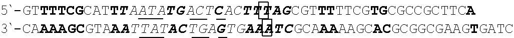 | 9 substitutions (1 sensitive) |
| Tn-T7.5 | 107 and 108 | 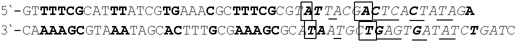 | 12 substitutions (3 sensitive) |
| Tn-T7.6 | 109 and 110 | 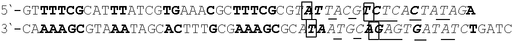 | 12 substitutions (2 sensitive) |
| Tn-T7.7 | 111 and 112 | 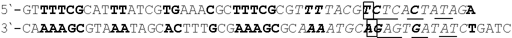 | 9 substitutions (1 sensitive) |
| Tn-T7.8 | 113 and 114 | 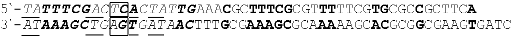 | 9 substitutions (2 sensitive) |

MuA transpososomes containing modified ends Tn-T7.1, Tn-T7.3, Tn-T7.4, Tn-T7.6, Tn-T7.7 and Tn-T7.8 were prepared and their activity was tested as described in Example 5. All tested complexes were able to fragment DNA, the activity of transposome complexes being similar to the activity as shown with complexes in FIGS. 13A-13C. Tn-T7.1, Tn-T7.3 as well as Tn-T7.4 showed the best level of activity among tested variants.

To confirm the functionality of T7 promoter within the transposon, Escherichia coli genomic DNA was fragmented using transpososomes containing either Tn-T7.1 or Tn-T7.3 modified ends. Upon cleanup, DNA was subjected to in vitro transcription (IVT) reaction containing 1× TranscriptAid™ reaction buffer, NTP mix (40 mM each), and 2 μl of TranscriptAid™ Enzyme Mix (Thermo Scientific) in 50 μl final volume. IVT was performed at 37° C. for 3.5 hours. To remove template DNA, the reaction mixtures were treated with DNase I. IVT products were then purified and analyzed on Agilent 2100 Bioanalyzer using the RNA 6000 Nano Kit (Agilent Technologies). RNA fragments were visible on the electropherogram confirming the success of IVT reaction. Obtained RNA fragment size distribution was in good agreement with the initial distribution of DNA fragments which were used as templates.

### Example 7. Transposon Ends Containing Random Sequences for Detection of Rare Mutations

A. Polymerase Fidelity Measurement Using Transposon Ends Containing Random Sequences

UMI tagmentation to incorporate barcodes using randomized transposon ends can be used to detect rare mutations by reducing sequencing background. Transposome complexes comprising transposon end nucleic acids with 12 randomized positions (SEQ ID NO: 16) were used to quantify erroneous substitutions by a high-fidelity proofreading DNA polymerase.

Sixteen PCR cycles were performed to amplify a 3.9 kb target from 1 ng of pPink-HC plasmid (from Invitrogen™ PichiaPink™ Vector Kit Catalog number: A11152) with a polymerase of interest according to recommendations provided by manufacturer. Forward and reverse primers were 5′-CCCACATCCGCTCTAACCGA (SEQ ID NO: 78) and 5′-CCCCGCATAAACACCTCTCTT (SEQ ID NO: 79), respectively. PCR product was purified from reaction mixture using the Collibri™ DNA Library Cleanup Kit (Invitrogen, Cat. No. A38584096). 50 μL of PCR reaction was mixed with 50 μL of magnetic beads and incubated for 5 min at room temperature. After a short spin, tubes were placed in a magnetic rack until the solutions were cleared. The supernatant was aspirated carefully without disturbing the beads and discarded. The beads were washed twice by incubating for 30 seconds with 200 μL 85% ethanol and removing the supernatant after 30 seconds of incubation. The tubes were given a short spin to collect excess ethanol and placed back into magnetic rack. Excess ethanol was removed, the beads were then air-dried by opening the tube caps for two minutes, allowing remaining ethanol to evaporate. The tubes were removed from the magnetic rack, the beads were resuspended in 17 μL of elution buffer (10 mM Tris-HCl (pH 8.3)) and placed back into magnetic rack. DNA was eluted by carefully aspirating the supernatant, and the DNA concentration was measured by NanoDrop spectrophotometer.

25 ng of purified PCR product was premixed with 2 μl of MuA complex in 1× Fragmentation Reaction Buffer (Thermo Scientific™ MuSeek™ Library Preparation Kit, Illumina™ compatible, Cat. No. K1361). Fragmentation was carried out in 30 μl reactions for 5 minutes at 30° C., then stopped by adding 3 μl of 4.4% SDS solution. Intact pPink-HC plasmid was fragmented as PCR-free control. Fragmented DNA was subjected to size selection using the Collibri™ DNA Library Cleanup Kit (Invitrogen, Cat. No. A38584096). The sample was mixed with 50 μl of magnetic beads and incubated for 5 min at room temperature. After a short spin, tubes were placed in a magnetic rack until the solutions were cleared. The supernatant was aspirated carefully without disturbing the beads and discarded. The beads were resuspended in 102 μl of elution buffer and placed back into magnetic rack until the solutions were cleared. 100 μl of supernatant was transferred in a new tube, mixed with 60 μl of magnetic beads, and incubated for 5 min at room temperature. After a short spin, the tubes were placed in a magnetic rack until the solutions were cleared. Supernatant was transferred in a new tube, mixed with 25 μl of magnetic beads, and incubated for 5 min at room temperature. After a short spin, the tubes were placed in a magnetic rack until the solutions were cleared. The supernatant was aspirated carefully without disturbing the beads and discarded. The beads were washed twice by incubating for 30 seconds with 200 μL 85% ethanol followed by removing the supernatant after 30 seconds of incubation. The tubes were given a short spin to collect excess ethanol and placed back into magnetic rack. Excess ethanol was removed, the beads were then air-dried by opening the tube caps for two minutes, allowing the remaining ethanol to evaporate. The tubes were removed from the magnetic rack, the beads were resuspended in 25 μL of elution buffer (10 mM Tris-HCl (pH 8.3)) and placed back into magnetic rack. DNA was eluted by carefully aspirating the supernatant.

Primers were designed to anneal to the transposon end nucleic acid sequence directly upstream of the N12 randomized sequence. Fragmented DNA containing random sequences was subjected to PCR amplification using Collibri Library Amplification Master Mix (Invitrogen, Cat. No. A38539050) to introduce Illumina P5/P7 adapters and library barcodes using the following primers:

P5-D501 (SEQ ID NO: 80):
AATGATACGGCGACCACCGAGATCTACACTATAGCCTATGCGACACTCGT
GAAACGCTTTCGCGTTT
P5-D502 (SEQ ID NO: 81):
AATGATACGGCGACCACCGAGATCTACACATAGAGGCATGCGACACTCGT
GAAACGCTTTCGCGTTT
P5-D503(SEQ ID NO: 82):
AATGATACGGCGACCACCGAGATCTACACCCTATCCTATGCGACACTCGT
GAAACGCTTTCGCGTTT
P7-D701(SEQ ID NO: 83):
CAAGCAGAAGACGGCATACGAGATATTACTCGCGAGGTCGAGTGCATGAA
ACGCTTTCGCGTTT
P7-D702(SEQ ID NO: 84):
CAAGCAGAAGACGGCATACGAGATTCCGGAGACGAGGTCGAGTCATGAAA
CGCTTTCGCGTTT
P7-D703(SEQ1DNO: 85):
CAAGCAGAAGACGGCATACGAGATCGCTCATTCGAGGTCGAGTGCATGAA
ACGCTTTCGCGTTT

A minimal amount of template (0.05 μL) was taken for amplification. The cycling protocol was: 1 cycle for 3 min at 66° C.; 1 cycle for 30 sec at 98° C.; 20 cycles for 15 sec at 98° C.; 30 sec at 60° C.; 30 sec at 72° C.; 1 cycle for 1 min at 72° C. Amplified library was purified from reaction mixture using the Collibri™ DNA Library Cleanup Kit. (Invitrogen, Cat. No. A38584096). 50 μL of PCR reaction was mixed with 40 μL of magnetic beads and incubated for 5 min at room temperature. After a short spin, the tubes were placed in a magnetic rack until the solutions were cleared. The supernatant was aspirated carefully without disturbing the beads and discarded. The beads were resuspended in 50 μL of elution buffer (10 mM Tris-HCl (pH 8.3)), and mixed with 50 μL of fresh magnetic beads. After a short spin and incubation for 5 min at room temperature, the tubes were placed in a magnetic rack until the solutions were cleared. The supernatant was aspirated carefully without disturbing the beads and discarded. The beads were washed twice by incubating for 30 seconds with 200 μL 85% ethanol and removing the supernatant after 30 seconds of incubation. The tubes were given a short spin to collect excess ethanol and placed back into magnetic rack. Excess ethanol was removed, the beads were then air-dried by opening the tube caps for two minutes, allowing remaining ethanol to evaporate. The tubes were removed from the magnetic rack, the beads were resuspended in 22 μL of elution buffer (10 mM Tris-HCl (pH 8.3)) and placed back into magnetic rack. DNA was eluted by carefully aspiring the supernatant. Agilent analysis and qPCR using Collibri Library Quantification Kit (Invitrogen, Cat. No. A38524500) were performed for library quality assessment. Libraries were pooled and sequenced on MiSeq instrument in paired 150 bp mode using custom primers:

Read 1: (SEQ ID NO: 86):
ATGCGACACTCGTTCGTGCGTCAGTTCA
Read 2: (SEQ ID NO: 87):
CGAGGTCGAGTGCAGTTCGTGCGTCAGTTCA
Index read: (SEQ ID NO: 88):
TGAACTGACGCACGAACTGCACTCGACCTCG

Generated sequencing data were analyzed by grouping reads to barcode (UMI) families and then calling polymerase errors. First, barcode sequences were extracted from reads using UMI-tools (v0.5.3). Next, adapters and low-quality sequences were trimmed using BBMAP (v37.17). Resulting reads were aligned with BWA aligner (v0.7.15) and grouped to families using UMI-tools group adjacency algorithm with hamming distance 1 (v0.5.3). Polymerase errors were called only if they are present in all reads in the UMI family, otherwise they were discarded as sequencing error.

Approximately 4 million unique barcode (UMI) sequences were observed within the sequencing data. A higher number (approximately 16 million) of unique sequences could theoretically be generated by recombinant transposon end sequences comprising substitutions at 12 positions, but the experimental data take into account reasons why some barcodes might not be found in the sequencing data. For example, only a fraction of fragmented DNA was harvested during size selection (so a substantial fraction of fragments was discarded for being outside the size selection boundaries) and only part of the constructed library was loaded onto the sequencing cell. Therefore, the experimental results indicate that the present methods with mixtures of recombinant transposon ends can generate a very large number of unique barcodes in NGS protocols. Further, the results suggest that this very large number of unique barcodes is of value because a substantial fraction of fragments labeled with barcodes will be lost during processes of size selection and sequencing. Also, the number of unique sequences/barcodes that can be introduced always has to be higher than the number of DNA fragments generated, to make sure that each fragment is barcoded uniquely.

Introduction of barcodes from transposome ends identified errors introduced by Platinum SuperFi DNA polymerase, which has a reported fidelity of >100×Taq. UMI tagmentation using randomized transposon ends reduced sequencing background (PCR free values) and revealed that Platinum SuperFi DNA polymerase had >300× greater fidelity compared to Taq, as shown in Table 5.

TABLE 5
Measurements of polymerase fidelity

| | Bases | Errors | | Polymerase | Polymerase |
|---|---|---|---|---|---|
| Sample | sequenced | found | Error rate | error rate* | fidelity** |
| Platinum | 4.51E+07 | 66 | 1.5E−06 | 1.72E−07 | 251 |
| SuperFi | | | | | |
| Platinum | 2.47E+07 | 20 | 8.1E−07 | 7.74E−08 | 559 |
| SuperFi | | | | | |
| Platinum | 9.47E+07 | 107 | 1.1E−06 | 1.27E−07 | 342 |
| SuperFi | | | | | |
| Platinum | 6.55E+07 | 67 | 1.0E−06 | 1.10E−07 | 393 |
| SuperFi | | | | | |
| Taq | 6.75E+07 | 20044 | 3.0E−04 | 4.67E−05 | 1× |
| Taq | 4.44E+07 | 12801 | 2.9E−04 | 4.61E−05 | 1× |
| PCR free | 7.71E+06 | 1 | 1.30E−07 | N/a | N/a |
| PCR free | 5.28E+06 | 2 | 3.79E−07 | N/a | N/a |

*Taking into account error accumulation during PCR.
**Normalized to Taq DNA polymerase

B. Detection of Low-Frequency Mutations

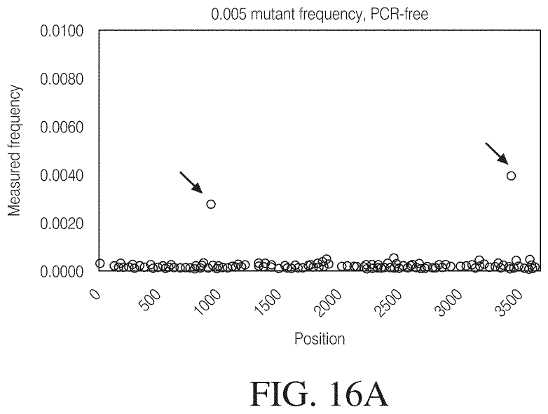

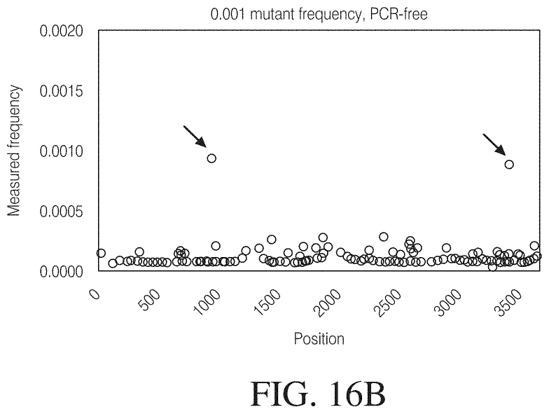

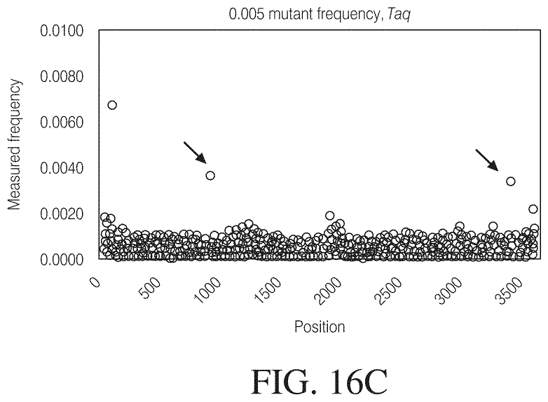

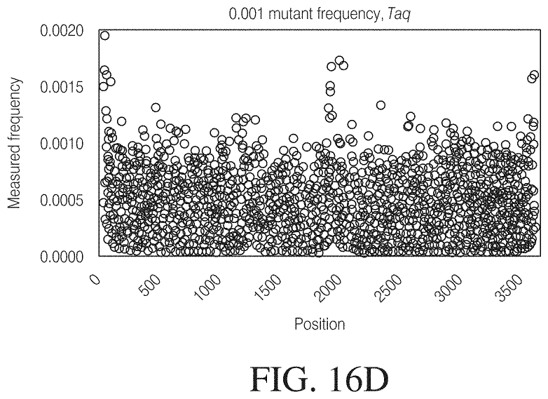

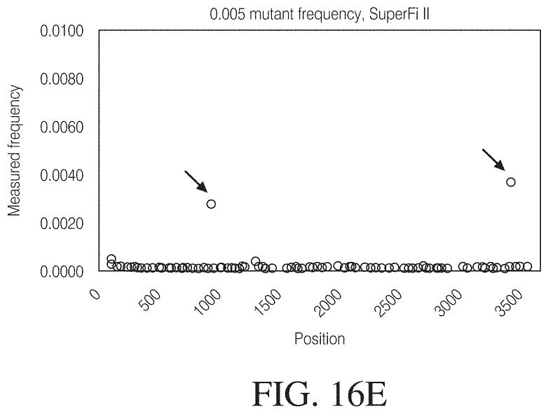

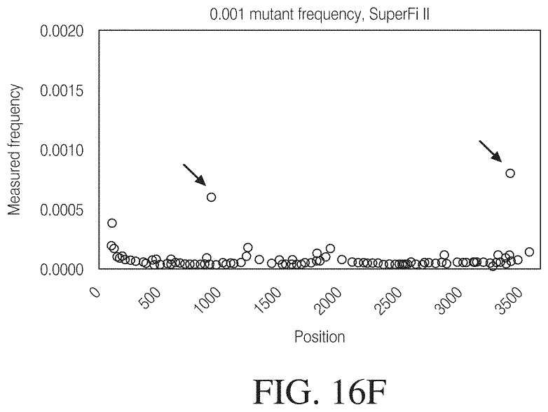

To demonstrate the feasibility of low-frequency mutation detection, two point mutations (A940G and T3428G) were introduced into a plasmid pPink-HC template. Then the mutant (pPink-HC with A940G and T3428G mutations) and wild-type (pPink-HC) plasmids were mixed at quantitative ratios of 1:200, 1:1000, and 1:5000 that simulate 0.005, 0.001, and 0.0002 mutation frequency, respectively. For each of the mixtures, a library was prepared using the transposome complexes comprising transposon end nucleic acids with 12 randomized positions (SEQ ID NO: 16) and sequenced. About 10 million of reads were obtained resulting in ˜10 000× coverage. The data was analyzed in analogy to Example 7A. Both mutations were detected at close to expected 0.005 and 0.001 frequency (FIG. 16A), while at 0.0002 frequency the mutations could not be confidently detected at this coverage. PCR amplification of the target region would allow rare-mutation detection in high-complexity DNA templates; however, uneven amplification of molecules may introduce discrepancies that make rare mutation detection even more complicated. To evaluate if the tagmentation with transposon ends with UMIs approach allows detection of rare mutation after preamplification, the same experiment was performed after the target region was amplified from wild-type/mutant plamid mixtures at quantitative ratios of 1:200, 1:1000 using either Taq or proofreading Platinum SuperFi II DNA polymerase. Preamplification of a 3.75 kb region by Taq DNA polymerase (GMP grade, Sigma-Aldrich) or Platinum SuperFi II was performed from 1 ng of plasmid DNA using primers 5′-CCCACATCCGCTCTAACCGA (SEQ ID NO: 91) and 5′-CCCCGCATAAACACCTCTCTT (SEQ ID NO: 92). Both mutations were detected after preamplification at close to expected frequency; however, the plot of all detected variants across the target region revealed a noisy background introduced by Taq DNA polymerase, and the mutations at the rate of 0.001 were lost within it (FIG. 16B). In contrast, Platinum SuperFi II DNA polymerase produced a negligible background (FIG. 16C), and mutations at the rate of 0.001 could be clearly detected after preamplification. The above experiments indicate that using tagmentation with transposon ends with UMIs approach greatly reduces the sequencing-related errors and allows detection of low-frequency mutations that are either present in the DNA sample or introduced during PCR preamplification.

For chromosome variant detection, multiplex PCR was performed using 1 ng of the structural multiplex reference standard (HD753, Horizon Discovery) genomic DNA and Platinum SuperFi II DNA polymerase. Primer sequences were the following: 5′-GCGAGTGACGCTTGGTGAA (SEQ ID NO: 93), 5′-GGAACCAGGGGTAGGTGATGA (SEQ ID NO: 94) (to amplify 756 bp from GNA11); 5′-CAGCCAGTGCTTGTTGCTTG (SEQ ID NO: 95), 5′-CCCTAGACAGGGAGTGCGAT (SEQ ID NO: 96) (to amplify 895 bp from AKT1); 5′-ACAAATTTCTACCCTCTCACGA (SEQ ID NO: 97), 5′-CTTTGAGAGCCTTTAGCCGC (SEQ ID NO: 98) (to amplify 720 bp from KRAS), 5′-CCAGTGCCCACTCAAGTCAT (SEQ ID NO: 99), 5′-AGGTGGACATCGATGAGTGC (SEQ ID NO: 100) (to amplify 822 bp from NOTCHI, and 5′-GGTGTCTAGCTGTCAGTGGT (SEQ ID NO: 101), 5′-TGTCGTTCACACAGCCAGAA (SEQ ID NO: 102) (to amplify 945 bp from FBXW7). The cycling protocol was 1 cycle—30 sec at 98° C.; 30 cycles—10 sec at 98° C.; 10 sec at 60° C.; 30 sec at 72° C.; 1 cycle—1 min at 72° C.

PCR products were purified from reaction mixtures using the Invitrogen™ Collibri™ DNA Library Cleanup Kit (Thermo Scientific), and concentrations were measured by the NanoDrop spectrophotometer. PCR products were subjected to NGS library preparation as described in previous examples, by using the tagmentation with transposon ends with UMIs approach. ˜10 million of reads were obtained resulting in ˜30 000× coverage, which was distributed evenly among the targets. All the targeted chromosome variants were confidently detected, although measured frequencies were slightly lower than expected (Table 6). These results indicate that the combination of multiplex PCR with the tagmentation with transposon ends with UMIs approach can be applied to detect sequence variants in high complexity DNA sequences.

TABLE 6
Genomic DNA variant detection

| | | Expected | Measured |
|---|---|---|---|
| Chromosome variant | Mutation | frequency | frequency |
| g.chr4:153244156_ | FBXW7 G667fs | 0.056 | 0.029 |
| 153244156delC | | | |
| g.chr9:139409754G > A | NOTCH1 P668S | 0.050 | 0.045 |
| g.chr12:25398281C > T | KRAS G13D | 0.056 | 0.034 |
| g.chr14:86246551C > T | AKT1 E17K | 0.050 | 0.031 |
| g.chr19:3118942A > T | GNA11 Q209L | 0.056 | 0.006 |

These data indicated that introduction of barcodes via a mixture of recombinant transposon end nucleic acid can significantly improve NGS error rate by reducing sequencing background.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A composition comprising a mixture of at least 25 different recombinant transposon end nucleic acids each independently comprising the nucleotide sequence of 5′-NNTTTCGNNNTTNNNNTGNNNCNNTTTCGNNNTTNNNNTGNNNCNNNNNA-3′ (SEQ ID NO: 20); wherein in each nucleic acid each N is independently chosen from A, C, G, and T.

2. The composition of claim 1, wherein the mixture of recombinant transposon end nucleic acids comprises the nucleotide sequence of 5′-NNTTTCGNNNTTNNNNTGNNNCNNTTTCGCGTTTNNNNTGNNNCNNNNNA-3′ (SEQ ID NO: 66); wherein in each nucleic acid each N is independently chosen from A, C, G, and T.

3. The composition of claim 1, wherein the mixture of recombinant transposon end nucleic acids comprises the nucleotide sequence of 5′-NNTTTCGNNNTTNNNNTGNNNCNNTTTCGCGTTTTTCGTGNNNCNNNNNA-3′ (SEQ ID NO: 67); wherein in each nucleic acid each N is independently chosen from A, C, G, and T.

4. The composition of claim 1, wherein the mixture of recombinant transposon end nucleic acids comprises the nucleotide sequence of 5′-NNTTTCGNNNTTNNNNTGNNNCNNTTTCGCGTTTTTCGTGCGCCNNNNNA-3′ (SEQ ID NO: 68); wherein in each nucleic acid each N is independently chosen from A, C, G, and T.

5. The composition of claim 1, wherein the mixture of recombinant transposon end nucleic acids comprises the nucleotide sequence of 5′-NNTTTCGNNNTTNNNNTGNNNCNNTTTCGCGTTTTTCGTGCGCCGCTTCA-3′ (SEQ ID NO: 69); wherein in each nucleic acid each N is independently chosen from A, C, G, and T.

6. The composition of claim 1, wherein the mixture of recombinant transposon end nucleic acids comprises the nucleotide sequence of 5′-GTTTTCGCATTTATCGTGAAACGCTTTCGNNNTTNNNNTGNNNCNNNNNA-3′ (SEQ ID NO: 74); wherein in each nucleic acid each N is independently chosen from A, C, G, and T.

7. The composition of claim 1, wherein the mixture of recombinant transposon end nucleic acids comprises the nucleotide sequence of 5′-GTTTTCGCATTTATCGTGAAACGCTTTCGCGTTTNNNNTGNNNCNNNNNA-3′ (SEQ ID NO: 16); wherein in each nucleic acid each N is independently chosen from A, C, G, and T.

8. The composition of claim 1, wherein the mixture of recombinant transposon end nucleic acids comprises the nucleotide sequence of 5′-GTTTTCGCATTTATCGTGAAACGCTTTCGCGTTTTTCGTGNNNCNNNNNA-3′ (SEQ ID NO: 75); wherein in each nucleic acid each N is independently chosen from A, C, G, and T.

9. The composition of claim 1, wherein the mixture of recombinant transposon end nucleic acids comprises the nucleotide sequence of 5′-GTTTTCGCATTTATCGTGAAACGCTTTCGCGTTTTTCGTGCGCCNNNNNA-3′ (SEQ ID NO: 12); wherein in each nucleic acid each N is independently chosen from A, C, G, and T.

10. The composition of any one of claims 1 to 9, wherein at least one transposon end nucleic acid has a sequence that has a nucleotide substitution at one or more positions corresponding to positions selected from 1, 2, 8, 9, 10, 13, 14, 15, 16, 19, 20, 21, 23, 24, 37, 41, or 49 positions of SEQ ID NO: 1.

11. The composition of any one of claims 1 to 10, wherein each nucleic acid in the mixture is unique.

12. The composition of any one of claims 1 to 11, wherein the mixture comprises at least 25, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000, 11000, 12000, 13000, 14000, 15000, 16000, 17000, 18000, 19000, 20000, 25000, 50000, 75000, 100000, 200000, 300000, 400000, 500000, 600000, 700000, 800000, 900000, 1000000, or more transposon end nucleic acids.

13. A composition comprising at least one transposase and the mixture of recombinant transposon end nucleic acids of any one of claims 1 to 12.

14. A method of fragmenting a sample comprising nucleic acids, comprising contacting the sample with the composition of claim 13.

15. The method of claim 14, wherein the sample is obtained from one cell.

16. A method of generating a population of uniquely barcoded nucleic acid fragments from a sample comprising nucleic acids comprising contacting the sample with a composition of claim 13, wherein the composition comprises at least 25, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000, 11000, 12000, 13000, 14000, 15000, 16000, 17000, 18000, 19000, 20000, 25000, 50000, 75000, 100000, 200000, 300000, 400000, 500000, 600000, 700000, 800000, 900000, 1000000, or more transposon end nucleic acids with different sequences.

17. A method of generating a population of barcoded nucleic acid fragments from a sample comprising nucleic acids, wherein the method comprises contacting the sample with a composition of claim 13, wherein the transposon end nucleic acids barcode the nucleic acid fragments from the sample.

18. The method of any one of claims 14 to 17, further comprising sequencing the population of barcoded nucleic acid fragments, optionally followed by any of sequence assembly, mutation analysis, allele analysis, copy number analysis, and/or haplotype analysis.

19. The method of claim 18, wherein the sequences of the barcodes are used for realignment of sequences in haplotype analysis.

20. The method of any one of claims 14 to 19, wherein the sequences of the barcodes are used to identify unique fragments generated during fragmentation of the sample.

21. A recombinant transposon end nucleic acid comprising a variant of the nucleotide sequence of SEQ ID NO: 1 having:
a. nucleotide substitutions at one or more positions corresponding to positions selected from 1, 2, 8, 9, 10, 13, 14, 15, 16, 19, 20, 21, 23, 24, 37, 41, or 49 positions of SEQ ID NO: 1;
b. nucleotide substitution at positions 6, 11, 12, 17, 18, 22, 25, 26 and/or 28, and, optionally, one or more nucleotide substitutions at positions corresponding to N positions in SEQ ID NO: 76;
c. nucleotide substitution at positions 33, 39, 40, and/or 44, and, optionally, one or more nucleotide substitutions at positions corresponding to N positions in SEQ ID NO: 73;
d. nucleotide substitution at positions 11 and 12, and, optionally, one or more nucleotide substitutions at positions corresponding to N positions in SEQ ID NO: 76;
e. nucleotide substitutions at positions 6, 12, and 17, and, optionally, one or more nucleotide substitutions at positions corresponding to N positions in SEQ ID NO: 76;
f. nucleotide substitutions at positions 12, 18, 22, and 25, and, optionally, one or more nucleotide substitutions at positions corresponding to N positions in SEQ ID NO: 76;
g. nucleotide substitutions at positions 40, and, optionally, one or more nucleotide substitutions at positions corresponding to N positions in SEQ ID NO: 74;
h. nucleotide substitutions at positions 33 and 40, and, optionally, one or more nucleotide substitutions at positions corresponding to N positions in SEQ ID NO: 74;
i. nucleotide substitutions at positions 39, 40, and 44, and, optionally, one or more nucleotide substitutions at positions corresponding to N positions in SEQ ID NO: 74;
j. nucleotide substitutions at positions 33, 39, and 40, and, optionally, one or more nucleotide substitutions at positions corresponding to N positions in SEQ ID NO: 74;
k. nucleotide substitution at position 28, and, optionally, one or more nucleotide substitutions at positions corresponding to N positions in SEQ ID NO: 77;
l. nucleotide substitutions at positions 26, and 28, and, optionally, one or more nucleotide substitutions at positions corresponding to N positions in SEQ ID NO: 77;
m. nucleotide substitutions at positions 17, 26, and 28, and, optionally, one or more nucleotide substitutions at positions corresponding to N positions in SEQ ID NO: 77;
n. nucleotide substitutions at positions 33, 34, 39, and 40, and, optionally, one or more nucleotide substitutions at positions corresponding to N positions in SEQ ID NO: 16; or
o. nucleotide substitutions of any one of (a)-(n) above and further comprising one, two, three, four, or five additional nucleotide substitutions compared to the nucleotide sequence of SEQ ID NO: 1.

22. The recombinant transposon nucleic acid of claim 21, wherein the nucleotide substitutions generate an additional biological function in the recombinant transposon end nucleic acid.

23. The recombinant transposon end nucleic acid of claim 22, wherein the additional biological function comprises (i) a primer binding site; (ii) all or part of a restriction endonuclease recognition site; and/or (iii) all or part of a promoter sequence.

24. The recombinant transposon end nucleic acid of claim 23, wherein the additional biological function is a promoter sequence.

25. The recombinant transposon end nucleic acid of claim 24, wherein the promoter sequence is a T3 or T7 promoter.

26. The recombinant transposon end nucleic acid of any one of claims 21 to 25, further wherein the nucleotide substitutions generate one or more barcodes.

27. A composition comprising one or more transposase and the recombinant transposon end nucleic acid of any one of claims 21 to 26.

28. The composition of claim 27, further comprising one or more additional recombinant transposon end nucleic acid of any one of claims 21 to 26, wherein the recombinant transposon end nucleic acids have different nucleotide sequences.

29. A method of generating a population of nucleic acid fragments from a sample comprising nucleic acids, wherein the method comprises contacting the sample with one or more composition of any one of claims 26 to 27.
